(19) United States
(12) Patent Application Publication
(10) Pub. No.: US 2010/0280975 A1
(43) Pub. Date: Nov. 4, 2010

(54) SYSTEMS FOR CLINICAL TRIALS

(75) Inventors: Claude Michel Wischik, Aberdeen (GB); Damon Jude Wischik, London (GB); Roger Todd Staff, Aberdeen (GB); Alison Dorothy Murray, Aberdeen (GB)

Correspondence Address: FOLEY AND LARDNER LLP;SUITE 500, 3000 K STREET NW, WASHINGTON, DC, 20007, US

(73) Assignee: WisTa Laboratories Ltd.

(21) Appl. No.: 12/741,342

(22) PCT Filed: Nov. 5, 2008
(86) PCT No.: PCT/GB2008/003736

§ 371 Date: May 4, 2010

Related U.S. Application Data
(60) Provisional application No. 60/996,177, filed on Nov. 5, 2007.
(60) Provisional application No. 61/077,281, filed on Jul. 1, 2008.

(51) Int. Cl. G06Q 90/00; A61B 5/00

(52) U.S. Cl. 705/500; 600/300

(57) ABSTRACT

The invention provides methods and systems for assessing the efficacy of a pharmaceutical which is putatively disease modifying of a cognitive disorder, for use in the treatment or prophylaxis of that cognitive disorder, the method comprising the steps of: (1) stratifying a subject group into at least 2 sub-groups according to a baseline indicator of likely disease progression, (2) treating members of each subject group with the pharmaceutical for a treatment time frame, (3) deriving psychometric and optionally physiological outcome measures for each treated patient group, (4) comparing the outcomes at (3) with a comparator arm of said sub-groups which is optionally a placebo or minimal efficacy comparator arm, (5) using the comparison in (4) to derive an efficacy measure for the pharmaceutical. The methods and systems of the invention address problems such as low rate of decline over the treatment time-frame of patients who have mild-disease severity at baseline and biased withdrawal, particularly in the placebo/comparator treatment arm.

### TECHNICAL FIELD

The present invention relates generally to methods for use in assessing the efficacy of a pharmaceutical treatments for cognitive disorders, particularly in respect of physiological and psychometric outcomes for putatively disease-modifying treatments.

### BACKGROUND ART

Clinical Trials for Disease Modifying Treatments

3,7-diaminophenothiazine (DAPTZ) compounds including methylthioninium chloride (“MTC”) have previously been shown to inhibit tau protein aggregation and to disrupt the structure of PHFs, and reverse the proteolytic stability of the PHF core (see WO96/30766, F Hoffman-La Roche). Such compounds were disclosed for use in the treatment and prophylaxis of various diseases, including Alzheimer's Disease (AD) and Lewy body disease. The rationale for the potential efficacy of DAPTZ compounds in the treatment and prophylaxis of disorders such as AD is based on their ability to act on the primary neurofibrillary pathology of Alzheimer's disease discovered by Dr. Alois Alzheimer.

However clinical proof of disease-modifying efficacy is not straightforward. In particular, trials carried out to provide such proof are critically dependent on the behaviour during the clinical trial of subjects randomised to inactive or minimally active treatment arms. Since the potential effect of such treatments is to prevent the clinical decline that would otherwise be expected, rather than to produce an immediate symptomatic improvement, it is evident that the effect size which is calculated as the difference between placebo- and active-treated arms will depend critically on the degree of decline occurring in subjects randomised to the placebo-arm.

A disease-modifying treatment cannot become generally available without rigorous proof of efficacy by way randomised double-blinded parallel-design clinical trials. Such trials must prespecify the time-point after randomisation (in a prespecified patient population/grouping) where there must be a statistically significant difference between subjects randomised to active treatment at some specified dose and subjects receiving either placebo or some minimally active comparator dose. An agreed outcome measure or measures must be prespecified. Alternative designs, where it is unethical to withhold already existing treatments, involve randomisation to alternative active treatment arms either singly or in some prespecified combination.

While these matters are generally well known in the prior art, and there have been numerous examples of clinical trials in cognitive disorders using treatment approaches which produce a temporary symptomatic boost in cognitive functioning (for example acetylcholine esterase inhibitors such Aricept, Reminyl, Exelon) there have been no examples to date in which there has been robust and unequivocal demonstration of disease-modification efficacy in clinical trials, which have not required post-hoc statistical adjustments to support the efficacy case. In general, while such post hoc analyses provide at best a plausible basis on which to conduct future confirmatory clinical trials, they do not themselves satisfy the requirements for provision of the compelling evidential burden favouring efficacy required to meet generally applicable regulatory standards, and thereby to permit such treatments to be generally prescribed.

There are considerable technical difficulties that are encountered in the conduct of trials aiming to demonstrate disease-modification efficacy. These are perhaps most clearly described by means of a concrete example. A 50-week Phase 2 exploratory dose-range-finding study (the “rember™ study”) for treatment of mild and moderate dementia of the Alzheimer type has been conducted using an investigational medicinal product (IMP) of which MTC was the active pharmaceutical ingredient (API). The study was a randomized, double blinded, placebo-controlled study whose primary objective was to investigate the effects of MTC at three doses (30, 60 and 100 mg, each three times per day), compared with placebo, on cognitive ability (as measured by the Alzheimer's Disease Assessment Scale—cognitive subscale; ADAS-cog).

The trial was planned on the basis of the general assumption in the field that subjects identified as having mild or moderate AD according to well-defined criteria generally accepted in the field (e.g. National Institute of Neurological and Communicative Disorders and Stroke—Alzheimer's Disease and Related Disorders Association [NINCDS-ADRDA] and Diagnostic and Statistical Manual of Mental Disorders, 4th Edn [DSMIV]) could be expected to decline over a 24-week study period when randomised to a placebo-treatment arm.

This assumption was in turn based on previous literature (e.g., Stern, R. G., Mohs, R. C., Davidson, M., Schmeidler, J., Silverman, J., Kramer-Ginsberg, E., Searcey, T., Bierer, L., Davis, K. L. (1994) A longitudinal study of Alzheimer's disease: measurement, rate, and predictors of cognitive deterioration. American Journal of Psychiatry, 151:390-396.; Doraiswamy, P. M., Kaiser, L., Bieber, F., Garman, R. L. (2001) The Alzheimer's Disease Assessment Scale: evaluation of psychometric properties and patterns of cognitive decline in multicenter clinical trials of mild to moderate Alzheimer's disease. Alzheimer Disease and Associated Disorders, 15:174-183; Birks, J. (2006) Cholinesterase inhibitors for Alzheimer's disease. Cochrane Database Systematic Reviews (1): CD005593).

One important feature of the study was that disease severity was determined at randomisation using the CDR (Hughes, C. P., Berg, L., Danziger, W. L., Coben, L. A., Martin, R. L. (1982) A new clinical scale for the staging of dementia. British Journal of Psychiatry, 140:566-572; Morris, J. C. (1993) The Clinical Dementia Rating (CDR): Current version and scoring rules. Neurology, 43:2412-2414) informed by the short version of the CAMDEX (Roth, M., Tym, E., Mountjoy, C. Q., Huppert, F. A., Hendrie, H., Verma, S. & Goddard, R. (1986) CAMDEX. A standardised instrument for the diagnosis of mental disorder in the elderly with special reference to the early detection of dementia. British Journal of Psychiatry, 149:698-709). Thus subjects could be classified into “mild” and “moderate” AD sufferers at the outset of the trial.

Problems With Shorter Duration Trials

However, as described in the disclosure of the invention below, the assumption turned out to be false in the context of the study in question. In particular, subjects identified as having mild AD did not decline over a 24-week study period when randomised to a placebo-treatment arm.

This unexpected finding by the present inventors thereby highlighted a new problem in demonstrating disease modifying efficacy in mild and moderate AD, and similar cognitive disorders.

Indeed, there have been a number of prominent large-scale clinical trial failures in studies aiming to demonstrate therapeutic efficacy in MCI (Salloway, S., Ferris, S., Kluger, A., Goldman, R., Griesing, T., Kumar, D. & Richardson, S. (2004) Efficacy of donepezil in mild cognitive impairment—A randomized placebo-controlled trial. Neurology, 63:651-657; Johnson and Johnson Pharmaceutical Research and Development. Study synopsis: a randomized double-blind, placebo-controlled trial to evaluate the efficacy and safety of galantamine in subjects with mild cognitive impairment (MCI) clinically at risk for development of clinically probable Alzheimer's Disease (Protocol No. GAL-INT-18), Jun. 17, 2004. http://www.clinicalstudyresults.org/documents/company-study_{—}96_{—}2.pdf; Petersen, R. C., Thomas, R. G., Grundman, M., Bennett, D., Doody, R., Ferris, S., Galasko, D., Jin, S., Kaye, J., Levey, A., Pfeiffer, E., Sano, M., van Dyck, C. H., Thal, L. J. for the Alzheimer's Disease Cooperative Study Group. (2005) Vitamin E and donepezil for the treatment of mild cognitive impairment. New England Journal of Medicine 352, 2379-2388; Feldman, H. H., Ferris, S., Winblad, B., Sfikas, N., Mancione, L., He, Y., Tekin, S., Burns, A., Cummings, J., del Ser, T., Inzitari, D., Orgogozo, J.-M., Sauer, H., Scheltens, P., Scarpini, E., Herrmann, N., Farlow, M., Potkin, S., Charles, H. C., Fox, N. C., Lane, R. (2007) Effect of rivastigmine on delay to diagnosis of Alzheimer's disease from mild cognitive impairment: the InDDEx study. Lancet Neurology 6:501-512). These trials needed to be run for 2 years in order to achieve sufficient statistical power in order to permit a difference in the rate of conversion to full clinical AD to be detected. It is not known whether these failures were due to the inherent lack of therapeutic efficacy of the substances tested, or whether failure was due to a structural fault in trial design that was limited by failure of subjects to decline, as was found for mild AD in the rember™ study.

Problems With Longer Duration Trials

One possible obvious solution to the “non-decline” problem discovered by the present inventors might be to design studies of longer duration than 6 months, although this did not help for the MCI trials referred to above. From previous literature, the estimated transition time from the CDR-mild to the CDR-moderate stages of AD is approximately 1.8-2.8 years (Galasko, D., Edland, S. D., Morris, J. C., Clark, C., Mohs, R., Koss, E. (1995) The Consortium to Establish a Registry for Alzheimer's Disease (CERAD). Part XI. Clinical milestones in patients with Alzheimer's disease followed over 3 years. Neurology, 45:1451-1455; Hughes, C. P., Berg, L., Danziger, W. L., Coben, L. A., Martin, R. L. (1982) A new clinical scale for the staging of dementia. British Journal of Psychiatry, 140:566-572; Devanand, D. P., Jacobs, D. M., Tang, M. X., Del Castillo-Castaneda, C., Sano, M., Marder, K., Bell, K., Bylsma, F. W., Brandt, J., Albert, M., Stern, Y. (1997) The course of psychopathologic features in mild to moderate Alzheimer disease. Archives of General Psychiatry, 54:257-263; Daly, E., Zaitchik, D., Copeland, M., Schmahmann, J., Gunther, J., Albert, M. (2000) Predicting conversion to Alzheimer disease using standardized clinical information. Archives of Neurology, 57:675-680; Berg, L., Miller, J. P., Storandt, M., Duchek, J., Morris, J. C., Rubin, E. H., Burke, W. J., Coben, L. A. (1988) Mild senile dementia of the Alzheimer type: 2. Longitudinal assessment. Annals of Neurology, 23:477-484.). Differences in the rate of decline have been documented previously in studies that have used baseline Mini-Mental State Examination (MMSE) or ADAS-cog score, and more recently CDR (Adak, S., Illouz, K., Gorman, W., Tandon, R., Zimmerman, E. A., Guariglia, R., Moore, M. M., Kaye, J. A. (2004) Predicting the rate of cognitive decline in aging and early Alzheimer disease. Neurology, 63:108-114). However, a time-frame of 1 to 3 years is not practical for the conduct of a clinical trial. For example, long trial duration engenders the ethical problem of recruiting patients to a trial in which subjects have a chance of being treated with placebo for a year or longer, while there is widespread availability of acetylcholinesterase (AChE) inhibitor treatment as an alternative. Indeed, ethical concerns were raised by regulatory authorities in the UK during the planning of the rember™ study regarding the acceptability of denying patients access to AChE inhibitors (e.g. Aricept), for longer than 6 months. This meant that in the rember™ study, placebo patients had to be switched to an active arm after 6 months.

For the same reason, it was necessary to specify as an exclusion criterion subjects who were anticipated to have a definite indication for the commencement of AD-labelled drugs for the duration of the randomised treatment period of the trial. In practice, such a requirement restricted recruitment to subjects who clinicians believed would not decline rapidly during the period of the trial. However, this acts as a selection bias in favour of subjects in whom disease modification efficacy cannot in principle be demonstrated.

In addition, longer trials in which patients/carers perceive continuing deterioration and/or side effects arising from the treatment engenders the problem of non-random drop-out over time. This has proved to be a particular problem in evaluating the efficacy of AChE inhibitors, even in 6-months studies. Non-random drop-out can inflate the apparent effect size of ineffective drugs, particularly in ITT/LOCF analyses (Intention to Treat/Last Observation Carried Forward) where the last available observation is used to impute missing data. It has been suggested that such bias has been introduced systematically in the evaluation of AChE inhibitors and has been known for some time (Gray R, Stowe R L, Hills R K, Bentham P. (2001) Non-random drop-out bias: intention to treat or intention to cheat? Controlled Clinical Trials 22(suppl 1):38S-39S; Hills R, Gray R, Stowe R, Bentham P. (2002) Drop-out bias undermines findings of improved functionality with cholinesterase inhibitors. Neurobiology of Aging 23(suppl 1):Abstract 338; Lavori P W (1992) Clinical trials in psychiatry: should protocol deviation censor patient data? Neuropsychopharmacology, 6:39-48; Little R Yau L (1996) Intent to treat analysis for longitudinal studies with drop outs. Biometrics, 52:1324-1333.). Conversely, whereas the effect of “fit-survivor” bias using LOCF data imputation is thought to inflate apparent effect size for drugs such as the AChE inhibitors, the effect for a drug which aims to stabilise disease progression, such as rember™, was found to be a compression of apparent effect size, particularly at the 50-week time point. This is because non-random drop-out occurs early in the active arms for the AChE inhibitors, whereas it occurred late in the placebo arm of the rember™ trial. Thus subjects in the Least Exposed Dose arm who continued to decline withdrew from the study largely after 24 weeks. This could be seen most clearly in the apparent stabilisation of the moderate group in the minimal efficacy treatment arm after 24 weeks in the ITT/LOCF analysis, which was almost certainly a trial artefact due to withdrawal of declining subjects.

Defining Disease Modifying Treatments

In addition to these methodological issues, there is the larger philosophical question as to the definition of what constitutes disease-modifying treatment as distinct from symptomatic treatment in terms of the burden of evidence required to prove disease-modification (Vellas, B., Andrieu, S., Sampaio, C. & Wilcock, G. (2007) Disease-modifying trials in Alzheimer's disease: a European task force consensus. The Lancet Neurology, 6:56-62).

One view focuses on what happens when a patient is withdrawn from active treatment. Symptomatic agents defer the symptoms of the disease without affecting the fundamental disease process and do not change the rate of longer term decline after an initial period of treatment. If after withdrawal the patient reverts to where they would have been without treatment, the treatment is deemed to be symptomatic (Cummings, J. L. (2006) Challenges to demonstrating disease-modifying effects in Alzheimer's disease clinical trials. Alzheimer's and Dementia, 2:263-271).

In the closely related field of Parkinson's Disease (PD) research, another view is that the issue is synonymous with delayed-start design (Clarke, C. E. (2004) A “cure” for Parkinson's disease: Can neuroprotection be proven with current trial designs? Movement Disorders, 19:491-498). That is, if a patient randomised late to active treatment is never able to catch up with a patient randomised early to active treatment, then the treatment is deemed to modify disease.

A third view is that the concept of disease-modification is simply a statement concerning mechanism of action. However, in the PD field the discussions remain theoretical, as there are as yet no proven disease-modifying treatments, even in the mechanistic sense (Parkinson Study Group. (2004) A controlled, randomized, delayed-start study of rasagiline in early Parkinson disease. Archives of Neurology, 61:561-566). Whatever the design chosen, the outcome of the trial depends critically on the expected rate of decline in the placebo-treatment arm. A clinical trial for MEM-1003, a calcium channel modulator, failed to demonstrate efficacy recently where there was an improvement of 3.2 units from the baseline ADAS-cog score in the placebo arm (Memory Pharmaceuticals (2007) http://phx.corporate-ir.net/phoenix.zhtml?c=175500&p=irol-newsArticle&t=Regular&id=1062734&).

One way that may appear an obvious solution to the ethical difficulties is to use an active comparator design. This is a study design in which subjects who have been stabilised on existing AD-labelled treatment (for example with an AChE inhibitor such as Aricept) are then randomised either to additional treatment with a putative disease-stabilising treatment such as rember™ or to add-on placebo treatment. The problem with this design, as explained further below, is that evidence has emerged from the rember™ study that long-term survivors of AChE-inhibitor treatment represent a biased selection depleted of decliners, who are described erroneously as “responders”. Since the population remaining on active treatment at randomisation is a biased subset with a reduced residual rate of decline, irrespective of disease-severity at baseline, the problem of non-decline in subjects randomised to the placebo/comparator arm reasserts itself, and again prevents or diminishes the ability of the trial to demonstrate disease-modification efficacy. This has been a particular problem accounting for the failure of a number of recent studies aiming to demonstrate disease-modifying efficacy by a mechanism of action through the β-amyloid pathology of AD. As with the failure of the MCI studies, it is now not possible to know whether the failure of these trials was due to an inherent fault in the rationale (i.e. β-amyloid is in fact irrelevant to disease progression in AD), or whether there was a structural fault in the add-on design which prevented demonstration of efficacy.

It will be apparent that this state of affairs is highly unsatisfactory, and that it acts as a significant obstacle to adducing proof of efficacy of treatments which have the potential to modify disease progression in cognitive disorders such as AD, particularly at the early stages. It should be borne in mind that similar considerations also apply to the design of studies aim to demonstrate disease-modifying efficacy in Parkinson's disease. Although different end-points are used in such studies (typically the UPDRS scale to measure severity of PD-related symptoms), the essential problems are the same, as they derive from the intrinsically slow progression dynamics of neurodegenerative diseases.

### BRIEF DISCLOSURE OF THE INVENTION

As described above, demonstration of decline in subjects randomised to placebo-treatment arms is critical to demonstrating therapeutic efficacy of disease-modifying treatments for cognitive disorders and other progressive neurodegenerative disorders.

However there are essentially 3 main reasons why subjects randomised to placebo-treatment arms in studies of progressive neurodegenerative disorders may fail to decline:

1. As noted above, one surprising discovery of the present invention is that that the primary trial design assumption was false in the rember™ study, i.e. patients who were CDR-mild at baseline did not significantly decline, although patients who were CDR-moderate declined at a somewhat faster rate than expected. This made it impossible to demonstrate the therapeutic efficacy of rember™ in subjects who were CDR-mild at baseline.

The present inventors propose that in the early stages of disease, endogenous compensation mechanisms, referred to below as “cognitive reserve” in the case of cognitive disorders, may operate to mask ongoing disease progression, so that subjects may appear not to decline clinically. More specifically, the act of recording the performance of a patient using a standard cognitive assessment instrument provides the subject with a learning experience in respect of the test used which permits the subject to compensate behaviourally for ongoing neurodegeneration.

This has clear implications, for example, in studies aiming to examine modification of disease progression at earlier stages of AD, such as mild AD or mild cognitive impairment (MCI), since one cannot in principle demonstrate efficacy over a 24-week study period.

However, it is obviously desirable to be able to demonstrate therapeutic efficacy at early disease stages, particularly for a treatment such as rember™ whose primary mode of action is to prevent the neuronal destruction that would otherwise occur as a result of neurofibrillary degeneration, occurring particularly in the medial temporal lobe brain regions.

2. In later stages of disease, subjects and their carers perceiving continuing decline withdraw in a biased manner from the placebo-treatment arm, leaving a population enriched in non-decliners in the placebo/comparator treatment arm, making it more difficult to demonstrate a difference between active treatment and placebo/comparator treatment.

3. Subjects already stabilised on active symptomatic treatment represent a biased selection of a non-decliner population.

These biased selection and retention artefacts likewise raise problems in demonstrating modification of disease progression in these neurodegenerative diseases.

The inventors have analysed these causes of placebo-non-decline in a clinical trial setting and provide herein novel methods to circumvent this with a view to demonstrating that a treatment is disease-modifying in a neurodegenerative disease context notwithstanding these obstacles.

The present invention relates generally to methods for demonstrating disease-modifying efficacy of materials for use in the treatment or prophylaxis of diseases, for example cognitive disorders. In particular it relates to improved methods for demonstration of disease-modifying treatment efficacy in circumstances, such as the early stages of cognitive disorders, when there is no evidence of apparent clinical decline over a treatment time-frame that is viable for the conduct of clinical trials, and which may therefore prevent detection of therapeutic efficacy. It also relates to circumstances, such as later stages of cognitive disorders, when longer trials might be required, but there is biased withdrawal of subjects receiving placebo or minimally active treatments which may likewise prevent detection of therapeutic efficacy.

Thus the invention provides various methods for assessing the efficacy of an IMP (Investigational Medicinal Product) or pharmaceutical having putative disease-modifying effect for use in the treatment of a neurodegenerative disease (e.g. cognitive disorder), the methods comprising the steps of:

(1) stratifying a subject group into at least 2 sub-groups according to a predictive indicator of future disease progression, such as clinical disease severity,

(2) treating some of each sub-group with the pharmaceutical for a treatment time frame,

(3) deriving physiological and\or psychometric outcome measures for each sub-group,

(4) comparing the outcomes at (3) with a comparator arm of the sub-group,

(5) deriving an efficacy measure for the pharmaceutical for each patient group.

In the methods of the invention described below, measures are taken which are designed to mitigate or solve one or more of the three problems highlighted above as they may pertain to the subject groups in question—these measures include:

(a) duration of trial, as appropriate to disease severity and other factors considered below,

(b) preferred outcome measures e.g. assessment of pathological burden in the brain may be employed,

(c) appropriate statistical correction for non-random withdrawal leading to fit-survivor bias e.g. by use of the linear imputation method described herein.

### FIGURES

In the more detailed description of the invention and its practice below, the following non-limiting Figures are referred to:

FIG. 1. Disease progression over 24 weeks for subjects with CDR-severity of mild and moderate. Shaded lines indicate fits derived from a linear, mixed-effects model.

FIG. 2. Disease progression measured by ADAS-cog (A) or MMSE (B) over 50 weeks separated by CDR-severity into mild and moderate. Shaded lines indicate fits derived from a linear, mixed-effects model.

FIG. 3. Education serves as a proxy of brain reserve, as indicated by Raven's progressive matrices (RPM), a measure of cognitive intelligence. The change in cognitive function was measured over one year and an improvement was seen in those who had more than 9 years of schooling. Means are adjusted for premorbid intelligence, brain burden and gender (data from Staff et al. 2004).

FIG. 4. Scans indicating brain activity. Regions are indicated as being activated, with increasing blood flow (white areas), or deactivated, with decreased blood flow (black areas). A typical scan is compared from a subject aging successfully (i.e. without loss of brain matter) (A) with that from a “cognitive decliner” (B).

FIG. 5. Treatment response at 24 weeks for rember™ in mild and moderate subjects. ADAS-cog measured at intervals for subjects treated with placebo, low, 30 mg or 60 mg given three times daily.

FIG. 6. Regions of tau aggregation pathology in Alzheimer's disease. The extent of tau aggregation in AD in different brain regions is indicated by the amount of stippling; a greater degree of stippling indicates greater levels of pathology.

FIG. 7. Typical SPECT scan appearances for different diagnostic groups. Each set of images shows coronal (top left), sagittal (top right) and transaxial (bottom left) views. The upper panel of images shows the SPECT perfusion scan; areas of highest activity are seen as white. Areas of perfusion are normally identified by using colour scales and without these it is difficult to distinguish these areas from areas of inactivity. Regions with rCBF activity have been identified, therefore, in the lower panel. A “negative” blood flow image is shown in the lower panel (i.e. grey areas of activity increasing to white areas of highest perfusion. The image from a normal subject (A) shows bilaterally symmetrical activity on the SPECT perfusion images, with greatest activity in the cortical grey matter of frontal, temporal, parietal and occipital lobes. There are no deficits or regions of reduced uptake. The “possible” AD image (B) shows only posterior temporo-parietal defects which are more subtle, and no other deficits or reduced uptake in the remainder of the cortex. The “probable” AD image (C) shows a posterior defect in the temporo-parietal association cortex, which can sometimes be unilateral, and shows no other deficits or reduced uptake in the remainder of the cortex. The vascular dementia image (D) shows patchy perfusion defects corresponding to one or more known vascular territories, and excludes posterior temporo-parietal defects characteristic of AD. The mixed image (E) shows a combination of vascular and AD characteristics.

FIG. 8. Region of interest analysis, as axial, sagittal and coronal views. Regions are shown as: frontal cortex (1); parietal cortex (2); temporal cortex (3); occipital cortex (4) and cerebellum (5).

FIG. 9. IIT/OC regions of significant correlation between baseline ADAS-cog severity and baseline cerebral blood flow. These regions of correlation are shown as whitened areas; the greater the correlation, the whiter the area. Threshold set at p<0.001, corrected at p<0.05 for multiple comparisons, both cluster and voxel significance.

FIG. 10. IIT/OC locations of significant decline between baseline and visit 4 in subjects treated with placebo who were CDR-mild at baseline. These regions of decline are shown as whitened areas; the greater the decline, the whiter the area. SPM analysis shows regions where rCBF was significantly less in visit 4 than visit 1. Threshold for difference set at p<0.005, corrected at p<0.05 for multiple comparisons, both cluster and voxel significance.

FIG. 11. ITT/OC change in rCBF in subjects who were CDR-mild at baseline. Treatment effects for the 30/60 mg group with respect to placebo were significant in the regions marked with “*”. Treatment effects for the low (100 mg) group with respect to placebo were significant in the regions marked “#”. Brain regions are denoted as follows: RTL (right temporal lobe), RPL (right parietal lobe), ROL (right occipital lobe), RFL (right frontal lobe), LTL (left temporal lobe), LPL (left parietal lobe), LOL (left occipital lobe), LFL (left frontal lobe).

FIG. 12. ITT/OC locations of treatment-dependent difference in decline between baseline and visit 4 in CDR-mild subjects treated with placebo versus those with rember™ at 30/60 mg tid. These regions of difference are shown as whitened areas; the greater the difference, the whiter the area. Threshold for difference p<0.005, corrected p<0.05 for multiple comparisons, both voxel and cluster significance.

FIG. 13. ITT/OC locations of treatment-dependent difference in decline between baseline and visit 4 in CDR-mild subjects treated with placebo versus those with rember™ at the low (100 mg). These regions of difference are shown as whitened areas; the greater the difference, the whiter the area. Threshold for difference p<0.005, corrected p<0.05 for multiple comparisons, both voxel and cluster significance.

FIG. 14. ITT/OC locations of regions of significant correlation between change in rCBF and change in ADAS-cog. These regions of correlation are shown as whitened areas; the greater the correlation, the whiter the area. SPM analysis shows regions where the change from baseline to visit 4 in active-treated subjects was significantly correlated with change in ADAS-cog score from baseline to visit 5. Threshold for difference set at p<0.005, corrected at p<0.05 for multiple comparisons, both cluster and voxel significance.

FIG. 15. PET image of a coronal section showing improvement of glucose uptake in the temporal lobe after 4 months treatment with rember™ (60 mg tid). The image on the left is that of a subject at baseline (A), that on right is of the same subject after 4 months treatment (B). The arrows point to increased glucose uptake in the hippocampal formation/entorhinal cortex after treatment.

FIG. 16. ITT/LOCF ADAS-cog change from baseline and fitted curves in subjects who were CDR-mild and CDR-moderate at baseline. ADAS-cog measured at intervals for subjects treated with placebo, low, 30 mg or 60 mg given three times daily.

FIG. 17. Rate of decline by ADAS-cog (A) and MMSE (B) for subjects in the placebo-low arm who had already had been previously exposed to AD-labelled treatment or who had been untreated prior to the trial.

FIG. 18. IIT/OC locations of regions of significant difference in blood flow in subjects previously treated with AD-labelled drugs versus treatment-naive subjects. These regions of difference are shown as whitened areas; the greater the difference, the whiter the area. Threshold for difference p<0.005, corrected p<0.05 for multiple comparisons, both voxel and cluster significance.

FIG. 19. ITT/OC ADAS-cog change from baseline and fitted curves. Subjects who received placebo during the base study were switched to low (100 mg) during extension phase E1 and are designated “placlow”. The broad shaded line is the inferred placebo decline. The proximity of the fits for placlow and inferred placebo indicates the small effect size of the 100 mg dose over 24 weeks.

FIG. 20. Relationship between Braak stage and mean MMSE score, adapted from Mukaetova-Ladinska et al 2000. In this study, the data were grouped according to four clinical severity stages as determined using the Cambridge Mental Disorders of the Elderly Examination (CAMDEX) rating system. The corresponding clinical severity ratings based on MMSE cut-points used more conventionally are shown on same axis with MMSE score.

FIG. 21. Braak stage probability distribution by age derived from Ohm et al. (1995) and analysis by inventors.

FIG. 22. Expected number of persons at each Braak by age in the United States of America. Derived, with analysis from inventors, from Ohm et al. (1995) and the U.N. World Population Prospects Population Database, 2004.

FIG. 23. Cumulative number of individuals in the United States of America by Braak stage. Derived, with analysis from inventors, from Ohm et al. (1995) and the U.N. World Population Prospects Population Database, 2004.

FIG. 24. Relationship between Braak stage and cognitive impairment over time. Progression from Braak stage 1 to 6 is estimated to take approximately 50 years. The transition from MMSE score of 30 to less than 20 takes approximately 30 years after the transition to Braak stage 1 and occurs at approximately Braak stage 4.

FIG. 25. Relationship between cognitive impairment, accumulation of aggregated Tau and Tau-mediated neuronal destruction over time. This is shown for entorhinal cortex (e.r.c.) and hippocampus (hippo.), two early casualties of the disease process and neocortex (cortex), where PHF accumulation occurs much later and more slowly.

FIG. 26. Regions of significant increase in glucose uptake relative to baseline in subjects treated with rember™ (60 mg tid or 100 mg tid) for 18 weeks. t-values shown on scale thresholded at p<0.005. The cluster in the left medial temporal lobe are significant (p<0.05) after correction for multiple correction across the whole head (Voxel level). Both medial temporal lobe clusters are significant when the data was small volume corrected for locations in the medial temporal lobe only. t-value map shown superimposed on a PET template to show approximate locations of difference.

FIG. 27. Regions of difference in glucose uptake with respect to placebo in subjects treated with rember™ (60 mg tid or 100 mg tid) for 18 weeks. t-values shown on scale thresholded at p<0.005. The cluster in the left medial temporal lobe is significant (p<0.05) after correction for multiple correction across the whole head (Voxel level). Both medial temporal lobe clusters are significant when the data was small volume corrected for locations in the medial temporal lobe only. t-value map shown superimposed on a single MRI scan to show approximate locations of differences.

### DETAILED DISCLOSURE OF THE INVENTION

Some of the factors relevant to the practice of the present invention will now be discussed in more detail:

Thus in one aspect of the present invention there is provided a method for assessing the efficacy of a pharmaceutical for use in the treatment of a cognitive disorder, the method comprising the steps of:

(1) stratifying a subject group into at least 2 sub-groups according to a baseline indicator of likely disease progression, such as disease severity,

(2) treating members of each subject group with the pharmaceutical for a treatment time frame,

(3) deriving psychometric and optionally physiological outcome measures for each treated patient group,

(4) comparing the outcomes at (3) with a comparator arm of said sub-groups,

(5) using the comparison in (4) to derive an efficacy measure for the pharmaceutical.

The methods of the invention are generally concerned with clinical trials for testing a pharmaceutical (or putative pharmaceutical e.g. an investigational medicinal product (IMP)), although they may also be employed for managing therapy whereby new treatment regimes employing the pharmaceutical are being tested or compared for their efficacy.

Thus the methods herein may be used for performing a clinical trial, or for providing a system for performing said trial.

The methods are particularly suitable to providing evidence of clinical efficacy suitable for meeting appropriate regulatory standards for marketing e.g. as required by the US Food and Drug Administration (FDA) or European Agency for the Evaluation of Medicinal Products (EMEA).

Some elements of the methods of the invention will now be considered in more detail.

Disease Modifying Pharmaceuticals

In the methods of the present invention the pharmaceutical will generally be one which is putatively “disease modifying” as distinct from symptomatic in action. This putative property may be inferred at the outset, for example, on the basis of a known or expected effect on the etiology of the disorder in question. As discussed elsewhere herein, disease modification may also be inferred from clinical evidence, e.g. if after withdrawal from treatment the patient reverts to where they would have been without treatment, the treatment may be deemed to be symptomatic rather than disease-modifying. Alternatively if a patient randomised late to active treatment is never able to catch up with a patient randomised early to active treatment, then the treatment is deemed to modify disease.

The methods of the invention are particularly applicable to putative inhibitors of pathological protein aggregation, where the aggregation is associated with neurodegeneration. In such conditions, protein which is associated with the disease undergoes an induced conformational polymerisation interaction, i.e one in which a conformational change of the protein seeds the binding and aggregation of further protein molecules in a self-propagating manner. Once nucleation is initiated, an aggregation cascade may ensue which involves the induced conformational polymerisation of further protein molecules, which conformational change may render the aggregates more resistant to further proteolysis. The protein aggregates thus formed are thought to be a proximal cause of neurodegeneration, clinical dementia, and other pathological symptoms of this group of diseases.

Examples of such proteins include the tau protein, synuclein proteins. It is also considered by many in the art that β-amyloid falls into this class.

Inhibitors of the aggregation of such proteins (i.e. putative pharmaceutical treatments) are described, for example, in WO96/030766; WO02/055720; WO2007/110627; WO03/007933; WO2006/032879; WO2007/110629; prior filed (unpublished) U.S. 60/945,006; prior filed (unpublished) PCT/GB2007/002570. However the methods of the present invention are generally applicable to any pharmaceutical for use in the treatment of a neurodegenerative disorder.

Thus one example inhibitor to which the present invention applies is a DAPTZ compound such as MTC, as described in the above cross-referenced disclosures. Examples include DAPTZ compounds and analogs thereof, having any of the following formulae:

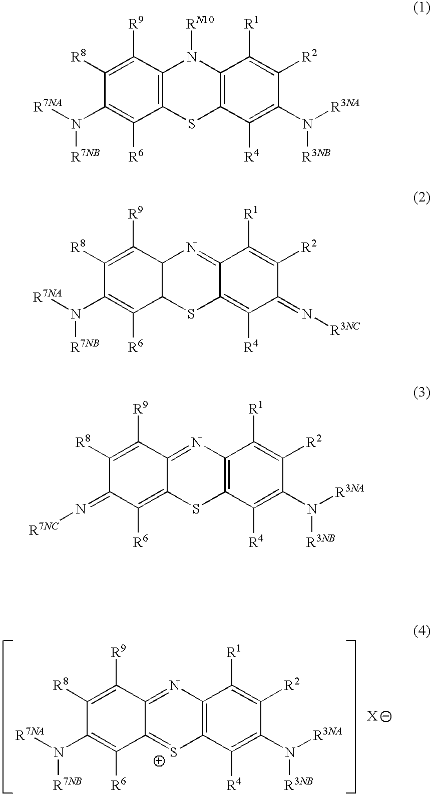

wherein each one of R¹, R², R⁴, R⁶, R⁸, and R⁹ is independently selected from:
—H;
-halogen (including —F; —Cl; —Br; —I)
—OH; —OR;
—SH; —SR;
—NO₂;
-carboxy (including: —C(═O)R)
—C(═O)OH; —C(═O)OR; —C(═O)NH₂; —C(═O)NHR; C(═O)NR₂; —C(═O)NR^{N1}R^{N2};)
—NH₂; —NHR; —NR₂; —NR^{N1}R^{N2};
   wherein in each group —NR^{N1}R^{N2}, independently, R^{N1} and R^{N2} taken together with the nitrogen atom to which they are attached form a ring having from 3 to 7 ring atoms;
—NHC(═O)H; —NRC(═O)H; —NHC(═O)R; —NRC(═O)R;
—R;
   wherein each R is independently selected from:
      substituted or unsubstituted alkyl or haloalkyl (including unsubstituted aliphatic C₁₋₆alkyl; substituted aliphatic C₁₋₆alkyl including halogenated alkyl);
      unsubstituted aliphatic C₂₋₆alkenyl; substituted aliphatic C₂₋₆alkenyl;
      unsubstituted C₃₋₆cycloalkyl; substituted C₃₋₆cycloalkyl;
      unsubstituted C₆₋₁₀carboaryl; substituted C₆₋₁₀carboaryl;
      unsubstituted C₅₋₁₀heteroaryl; substituted C₅₋₁₀heteroaryl;
      unsubstituted C₆₋₁₀carboaryl-C₁₋₄alkyl; substituted C₆₋₁₀carboaryl-C₁₋₄alkyl;

wherein,

in each group —NR^{3NA}R^{3NB}, if present, each one of R^{3NA} and R^{3NB} is independently H; —OH; carboxy; alkoxy; or as defined above for R; or R^{3NA} and R^{3NB} taken together with the nitrogen atom to which they are attached form a ring having from 3 to 7 ring atoms;

in each group —NR^{7NA}R^{7NB}, if present, each one of R^{7NA} and R^{7NB} is independently H; —OH; carboxy; alkoxy; or as defined above for R; or R^{7NA} and R^{7NB} taken together with the nitrogen atom to which they are attached form a ring having from 3 to 7 ring atoms;

in each group ═NR^{3NC}, if present, R^{3NC} is independently H; —OH; carboxy; alkoxy or as defined above for R;

in each group ═NR^{7NC}, if present, R^{7NC} is independently H; —OH; carboxy; alkoxy; or as defined above for R;

R^{N10}, if present, is independently H; —OH; carboxy; alkoxy; or as defined above for R;

X⁻, if present, is one or more anionic counter ions to achieve electrical neutrality.

and all pharmaceutically acceptable salts, hydrates, and solvates thereof. As shown above such compounds may be in oxidised or reduced form, and may be highly purified and in modified dosage forms. Thus, this includes (without limitation):
Methylthioninium [MT] and all salts thereof (including the mono-chloride salts and di-protic acid derivatives of leucoform or ‘free base’.
Ethylthioninium [ET] and all salts thereof (including chlorides, bromides and nitrates)
Diethylmethylthioninium and all salts thereof (including chloride [DEMTC])
Dimethylmethylthioninium and all salts thereof (including chloride [DMETC])
Diethylethylthioninium and all salts thereof (including chloride [DEETC])
Dimethylmethylthioninium and all salts thereof (including chloride [DMMTC])

However it will be understood that the disclosure herein is applicable to other disease modifying treatments.

Neurodegenerative Disorders

Example diseases to which the present methods may apply (e.g. which are characterised by pathological protein aggregation) include Alzheimer's disease, MCI, motor neurone disease, Fronto-temporal dementia and related so-called tauopathies and Lewy body disease. Furthermore, the pathogenesis of neurodegenerative disorders such as Pick's disease and Progressive Supranuclear Palsy appears to correlate with an accumulation of pathological truncated tau aggregates in the dentate gyrus and stellate pyramidal cells of the neocortex, respectively.

Typically the neurodegenerative disease is a cognitive disorder, most typically one where the progression dynamics are relatively slow.

Examples of cognitive disorders include mild and moderate AD, and MCI.

While there is still discussion in the literature as to the nature of the MCI concept (see Gauthier et al., Lancet, 2006; 367: 1262-1270; Petersen R C et al. Neuropathological features of amnestic mild cognitive impairment. Arch Neurol 2006; 63: 665-672) MCI is recognised as a valid disease target by the FDA. It is defined by having a minor degree of cognitive impairment not yet meeting clinical criteria for a diagnosis of dementia.

Representative criteria for syndromal MCI include features listed below:

A. The patient is neither normal nor demented.

B. There is evidence of cognitive deterioration shown by either objectively measured decline over time and/or subjective report of decline by self and/or informant in conjunction with objective cognitive tests (e.g. secondary tests if memory).

C. Activities of daily living are preserved and complex instrumental functions are either intact or minimally impaired.

(See also Winblad, B. et al. (2004) Mild cognitive impairment—beyond controversies, towards a concensus: report of the International Working Group on Mild Cognitive Impairment. J. Intern. Med. 256: 240-246).

As used above, the term “dementia” refers to a psychiatric condition in its broadest sense, as defined in American Psychiatric Association: Diagnostic and Statistical Manual of Mental Disorders, Fourth Edition, Washington, D.C., 1994 (“DSM-IV”). The DSM-IV defines “dementia” as characterized by multiple cognitive deficits that include impairments in memory and lists various dementias according to presumed etiology. The DSM-IV sets forth a generally accepted standard for such diagnosing, categorizing and treating of dementia and associated psychiatric disorders.

The MCI may be “amnestic”.

By one preferred definition, individuals with amnestic MCI have general cognitive measures within 0.5 standard deviations of control subjects and also have memory performance 1.5 standard deviations below control subjects. An objective, documented decline in memory is useful in determining which individuals have MCI.

“MCI-nonamnestic” or “MCI-other” may be defined as deficits in two or more areas of cognition greater than 1.5 standard deviations below the mean, corrected for age and education.

MCI subjects for whom the present invention may preferably be used may be those with less than or equal to MMSE 24,25,26,27,28 or 29, more preferably less than or equal to MMSE 24,25,26, most preferably less than or equal to MMSE 24 or 25.

In one aspect the disorder is Parkinson's disease. Although different end-points are used in such studies compared to AD (typically the UPDRS scale to measure severity of PD-related symptoms), the essential problems are the same, as they derive from the intrinsically slow progression of the disease.

Subject Groups and Predictive Indicators of Likely Disease Progression

The subject group will typically be patients diagnosed with the disorder in question using conventional criteria (e.g. National Institute of Neurological and Communicative Disorders and Stroke—Alzheimer's Disease and Related Disorders Association [NINCDS-ADRDA]; The American Psychiatric Association: Diagnostic and Statistical Manual of Mental Disorders, Fourth Edition, Washington, D.C., 1994 [“DSM-IV”].

The DSM-IV sets forth a generally accepted standard for such diagnosing, categorizing and treating of dementia and associated psychiatric disorders.

In the methods of the invention the subject group is itself stratified according to baseline indicators of likely disease progression. This in turn can be assessed in terms of disease severity.

Preferably, in AD, disease severity is assessed using the so-called Clinical Dementia Rating (CDR) scale (Hughes, C. P., Berg, L., Danziger, W. L., Coben, L. A., Martin, R. L. (1982) A new clinical scale for the staging of dementia. British Journal of Psychiatry, 140:566-572; Morris, J. C. (1993) The Clinical Dementia Rating (CDR): Current version and scoring rules. Neurology, 43:2412-2414).

The CDR may optionally be informed by a structured clinical examination e.g. the short version of the CAMDEX (Roth, M., Tym, E., Mountjoy, C. Q., Huppert, F. A., Hendrie, H., Verma, S. & Goddard, R. (1986) CAMDEX. A standardised instrument for the diagnosis of mental disorder in the elderly with special reference to the early detection of dementia. British Journal of Psychiatry, 149:698-709). Alternatively the CDR may be informed by the structured psychiatric interview as defined by Hughes et al. (1982) or Morris (1993).

For example, sub-groups may be formed from subjects having a CDR rating of 1 (mild sub-group) or 2 (moderate sub-group).

Disease severity may also be assessed e.g. using the “Braak staging” methods described in WO 02/075318. Sub-groups may then be formed from subjects having Braak stage up to 1, 2, 3 and 4, and so on.

The sub-groups will generally be tested in parallel.

It will be understood that the term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included, which in the context of this patent application may include the treatment of MCI or mild to AD with the intention of inhibiting the irreversible damage which occurs in the brain structures critical for memory function in later stages of AD.

Subjects may be selected from those not previously stabilised on active symptomatic treatment. Where such subjects are included in the trial, they should be randomised between treatment and comparator arms after first ascertaining their rate of decline on active symptomatic treatment by employing a run-in observation period prior to randomisation to disease-modifying treatment to ascertain actual rate of decline on symptomatic treatment.

Treatment Time Frame

As noted above, the present invention is particularly applicable to neurodegenerative diseases having relatively slow progression.

As described below, the treatment time frame can be selected based on the disease severity of the subgroup. Typical time frames for a clinical trial according to the present invention may be more than or equal to 12 weeks, 16 weeks, 24 weeks, 25 weeks, 36 weeks, 50 weeks, 100 weeks (or more than or equal to 3 months, 4 months, 6 months, 9 months, 12 months, 24 months and so on). Depending on the time frame thus selected, the present invention provides for the use of novel measures or analysis to derive more accurate measures of pharmaceutical efficacy.

Preferred trials may be less than the periods above, e.g. less than 9, 6, 5, 4, or 3 months.

For example for shorter time scales, and e.g. in patients having relatively low disease severity at baseline (where cognitive decline as measured by psychometric outcome measures may be masked by cognitive reserve) it may be preferable to use additional physiological outcome measures and/or more sensitive psychometric outcome measures.

Other trials may be more than 6 or 12 months.

For example for longer time scales (and e.g. in patients having relatively high disease severity at baseline, where “fit survivor” artifacts are more likely to occur), it may be preferable to use a linear imputation method for each individual discontinuing treatment to correct the analysis for the effect of discontinuation.

The time-frames may be same or different for the sub-groups.

Psychometric Outcome Measures

Psychometric outcome measures for use in the methods may be conventional ones, as accepted by appropriate regulatory bodies.

For AD, the Alzheimer's Disease Assessment Scale—cognitive subscale [ADAS-cog] is preferred Rosen W G, Mohs R C, Davis K L. A new rating scale for Alzheimer's disease. Am J Psychiatry. 1984 November; 141(11):1356-64).

Another standardised test is the Mini-Mental State Examination [MMSE] which was proposed as a simple and quickly administered method for grading cognitive function (Folstein M F, Folstein S E & McHugh P R. ‘Mini-mental state’. A practical method for grading the cognitive state of patients for the clinician. Journal of Psychiatric Research 1975 12 189-198.). The MMSE is the most widely used cognitive screening instrument for the detection of cognitive dysfunction due to dementia in geriatric and psychiatric patients (Tombaugh T N & McIntyre N J. The mini-mental state examination: a comprehensive review. Journal of the American Geriatric Society 1992 40 922-935). The MMSE evaluates orientation, memory, attention and language functions.

As described below, assessment or analysis of psychometric outcome measures may include the step of performing a linear imputation analysis on the available psychometric scores of individual subjects discontinuing treatment. This may involve a straight line per-subject extrapolation fitted to the graph of said scores (e.g. ADAS-cog change scores).

As described below, more sensitive psychometric measures may also be employed, and these may permit shorter testing intervals.

Physiological Outcome Measures

As described herein, in addition to psychometric testing, the present inventors provide for the use of neurophysiological outcome measures, e.g. by way of analysis of changes in functional brain scans. This increases the sensitivity of analysis of disease modifying treatment when testing even for relatively short time periods, e.g. 3 or 4 months.

Scans may employ SPECT (Single Photon Emission Tomography) with the ligand ^{99m}Tc-HMPAO, or reductions in cerebral glucose uptake as measured by PET (Positron Emission Tomography) using ¹⁸fluoro-deoxyglucose (FDG), in the temporo-parietal association neocortex in AD.

The use of such scans generally is well known in the art for diagnosis (see e.g. Talbot, P. R., Lloyd, J. J., Snowden, J. S., Neary, D., Testa, H. J. (1998) A clinical role for 99mTc-HMPAO SPECT in the investigation of dementia? Journal of Neurology, Neurosurgery and Psychiatry, 64:306-313.; Masdeu, J. C., Zubieta, J. L., Arbizu, J. (2005) Neuroimaging as a marker of the onset and progression of Alzheimer's disease. Journal of the Neurological Sciences, 236:55-64) and in examining response to therapy (Venneri, A., Shanks, M. F., Staff, R. T., Pestell, S. J., Forbes, K. E., Gemmell, H. G., Murray, A. D. (2002) Cerebral blood flow and cognitive responses to rivastigmine treatment in Alzheimer's disease. NeuroReport; 13:83-87).

It will be appreciated that, in addition to these techniques, any other applicable methodology which permits direct measure of pathological burden in the brain may be employed.

However the fact that such methods, hitherto generally used for diagnostic or other purposes in patients demonstrating cognitive changes in response to treatment or disease progression also have applicability in the detection of therapeutic efficacy even in circumstances where there is no apparent clinical benefit of a disease-modifying treatment, is highly unexpected.

Thus in the present invention, subjects will be scanned at or shortly before (preferably less than 3 months) the time of randomisation i.e. prior to treatment with the pharmaceutical being tested, or comparison treatment (placebo, or other dose or pharmaceutical).

One or more later scans will then be made after or during treatment, but preferably within 6 months.

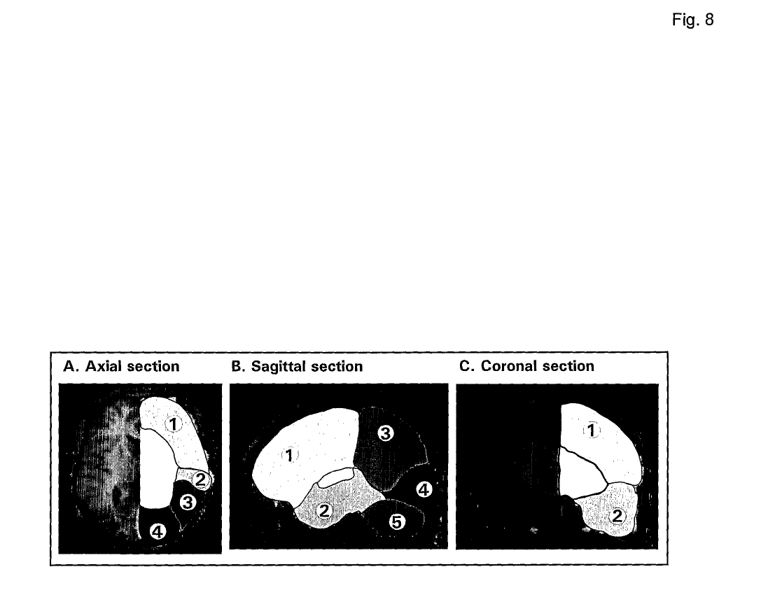

Thus in this embodiment the method comprises measurement of a change in functional brain scan in subjects treated with placebo or other comparator, using methods such as SPECT or PET, and comparing these subjects receiving active treatment. The effect of treatment can be demonstrated by either Region of Interest (ROI) Analysis or Statistical parametric (SPM) analysis. A standardised ROI may be created for each lobe of the brain and divided into hemispheres for frontal, parietal, temporal and occipital lobes (8 ROIs), and cerebellum (see e.g. FIG. 8). Counts derived from each of the first eight regions may be normalised with respect to counts in cerebellum to make allowance for inter-individual variation, and non-specific pharmacological effects of the pharmaceutical. These normalised counts for 8 ROI's may be further reduced to a single per-subject parameter calculated from a principal components analysis which provides a general per-subject factor and accounts separately for lobe-specific variance normalised to a value of 1 with a standard deviation of 0.15.

As shown in the Examples below, decline seen on the functional brain scans was predictive of future clinical decline that emerged six months later in the CDR-mild group. This strongly supports the conclusion that psychometric measures of disease progression are confounded by cognitive reserve capacity, particularly at the stage of the disease captured within the CDR-mild category.

Thus the physiological outcome measures of (3) may be any of those described above.

Comparator Arm

Following randomisation of the sub-groups, some subjects in each sub-group will be selected for comparator treatment. Preferably this will be a a placebo (non-treatment) or minimal efficacy comparator arm of the trial. However alternative designs, where it is unethical to withhold already existing treatments, involve randomisation to alternative active treatment arms either singly or in some prespecified combination.

Final Efficacy Measure

Typically this will ultimately be based on a psychometric outcome, based on appropriate statistical methods for comparing the relevant treatment and placebo arms. This may optionally be ANCOVA or if necessary to achieve more power a linear-mixed effects approach (Petkova, E. and Teresi, J. (2002) Some statistical issues in the analysis of data from longitudinal studies of elderly chronic care populations. Psychosomatic Medicine, 64:531-547) such as shown in Examples below. Physiological outcome measures may also be employed in the final analysis. Suitable clinical end points demonstrating efficacy in this respect can be selected by those skilled in the art, in the light of the present disclosure.

Specifically, efficacy can be demonstrated where there is a statistically significant difference between subjects randomised to active treatment at some specified dose and subjects receiving the comparator treatment, dose or placebo.

### Example Embodiments

Thus in one embodiment the subject group is stratified into mild AD (CDR=1) and moderate AD (CDR=2). Another sub-group may be Mild Cognitive Impairment (MCI as defined by agreed clinical criteria or CDR=0.5).

In the MCI and mild AD sub-group the time frame may be insufficient to expect, in the light of the results described herein, decline in the psychometric outcome measure (e.g. less than 6 months).

Thus physiological outcome measures are used in addition to psychometric outcome measures. These may be in the time frame up to 6 months for mild AD (e.g. 3 to 4 months), and even longer (6-12 months for MCI). As shown herein, efficacy demonstrated by such measures can predict future efficacy using clinical-psychometric end-points.

In mild or moderate AD, dosage strengths of a putative disease-modifying treatment, which may produce limited or no apparent therapeutic efficacy over 6 months, but which produces evidence of physiological efficacy over 6 months, can be expected to have clinical-psychometric therapeutic efficacy over 12 months.

In another embodiment, the sub-group (MCI, mild AD, most preferably moderate AD) and time frame are such as to expect, in the light of the results described herein, decline in the psychometric outcome measure. In mild AD defined by CDR, the trial should optimally be conducted for a period of 12 months in order to demonstrate a clinical end-point, such as a significant difference from placebo on a cognitive instrument such as ADAS-cog.

It will be apparent to those skilled in the prior art that the psychometric testing interval may be shorter if a more sensitive indicator of early cognitive decline is used such as a delayed match to sample procedure or paired-associates learning (Fowler, K S., Saling, M. M., Conway, E. L., Semple, J. S. & Louis, W. J. (1995) Computerised delayed matching-to-sample and paired associate performance in the early detection of dementia. Applied Neuropsychology 2: 72-78; Fowler, K. S., Saling, M. M., Conway, E. L., Semple, J. S. & Louis, W. J. (1997) Computerised neuropsychological tests in the early detection of dementia: prospective findings. Journal of the International Neuropsychological Society 3:139-146; Swainson, R., Hodges, J. R., Galton, C. J., Semple, J., Michael, A., Dunn, B. D., Iddon, J. L., Robbins, T. W. & Sahakian, B. J. (2001) Early detection and differential diagnosis of Alzheimer's disease and depression with neuropsychological tasks. Dementia and Geriatric Cognitive Disorders 12:265-280) or a dual-task paradigm (Blackwell, A. D., Sahakian, B. J., Vesey, R., Semple, J. M., Robbins, T. W. & Hodges, J. R. (2003) Detecting dementia: novel neuropsychological markers of preclinical Alzheimer's disease. Dementia and Geriatric Cognitive Disorders 17, 42-48) or other more complex psychometric batteries containing these as elements (eg CANTAB [Cambridge Computerised Neuropsychological Test Automated Battery], CANTAB PALT [CANTAB Paired Associates Learning], CNTB [Computerised Neuropsychological Test Battery], and Cognitive Drug Research Computerised Assessment System).

In moderate AD defined by CDR, the trial should optimally be conducted for 6 months, as a trial of longer duration risks non-random withdrawal of moderate subjects randomised to the placebo or minimal efficacy comparator treatment arm, thereby preventing or confounding the demonstration of therapeutic efficacy.

In moderate AD, where the trial is conducted over a period longer than 6 months with the intention of demonstrating that disease-modification efficacy is maintained on clinical-psychometric outcome measures, non-random withdrawal leading to fit-survivor bias can be corrected by the linear imputation method described herein without introducing bias and without inflation of the estimated effect size.

Thus in the methods described herein, wherein the time frame is sufficient to expect decline in the psychometric outcome measure but not short enough to mitigate the effect of non-random withdrawal, then such withdrawal is corrected by a linear imputation method described herein, rather than the more convention LOCF method. This aids correction of so called “fit-survivor bias”.

Thus wherein any subject discontinues treatment, the method employs a straight line extrapolation fitted to the graph of available psychometric scores (e.g. ADAS-cog change scores) against visit-date for the available data for that subject. Preferably, the line is not forced to pass through zero.

It should be noted that the linear imputation method may not be applicable in mild AD if no decline has been registered in the period—the imputed score derived from the early phases of the trial will describe a horizontal line. Therefore, in mild AD, it is necessary either to use a physical primary outcome measure (such as SPECT or PET described above) or to conduct the trial over a longer period or to use a psychometric instrument more sensitive to early stage decline.

As discussed above, patients stabilised on active symptomatic treatment are likely to represent a biased selected group who have a lower rate of decline due to prior withdrawal of subjects who experience continuing decline, often erroneously labelled at “non-responders”. In respect of these subjects, combinations of approaches described herein are preferred.

First, physical primary outcome measures based on repeated SPECT or PET scans as described above may be able to reveal ongoing decline on placebo and arrest of decline on active treatment with a disease-modifying drug.

Second, the incorporation of a run-in period of observation in subjects receiving only active symptomatic treatment prior to randomisation to disease-modifying drug or placebo will permit objective determination of expected rate of decline, and the linear imputation approach described herein can be used to document deviations from expected rate of decline due to ongoing non-random withdrawal of declining subjects in the placebo treatment arm.

Finally, as would be apparent to skilled practitioners in the field the incorporation of delayed-start or randomised-withdrawal (e.g. Clarke, C. E. (2004) A “cure” for Parkinson's disease: Can neuroprotection be proven with current trial designs? Movement Disorders 19, 491-498; Thal, L. J., Kantarci, K., Reiman, E. M., Klunk, W. E., Weiner, M. W., Zetterberg, H., Galasko, D., Pratico, D., Griffin, S., Schenk, D. & Siemers, E. (2006) The role of biomarkers in clinical trials for Alzheimer disease. Alzheimer Disease and Associated Disorders 20, 6-15) components to the study design will permit support for disease-modifying claims that would be acceptable to regulatory agencies.

Any sub-titles herein are included for convenience only, and are not to be construed as limiting the disclosure in any way.

The disclosure of all references cited herein, inasmuch as it may be used by those skilled in the art to carry out the invention, is hereby specifically incorporated herein by cross-reference.

### Example 1

### Disease Severity at Baseline and Rate of Disease Progression

It has been generally recognised in the literature that severity or stage of disease is an important predictor of disease progression (reviewed recently in Schaufele, M., Bickel, H., Weyerer, S. (2002) Which factors influence cognitive decline in older adults suffering from dementing disorders? International Journal of Geriatric Psychiatry, 17:1055-1063).

However the rember™ study is the first in which CDR severity at baseline was pre-specified as a stratification covariate in the primary outcome analysis. Subjects were classified into two groups: those who were CDR-mild at baseline (including 3 [1% of total randomised] who were CDR-questionable at baseline) and those who were CDR moderate at baseline. Although CDR has been advocated previously as a staging instrument (Berg, L., Danziger, W. L., Storandt, M., Coben, L. A., Gado, M., Hughes, C. P., Knesevich, J. W., Botwinick, J. (1984) Predictive features in mild senile dementia of the Alzheimer type. Neurology, 34:563-569), the more conventional approach to date has been to use baseline MMSE (e.g., MMSE≦19 vs. MMSE>19), or baseline ADAS-cog classifications (e.g. ADAS-cog>25 vs. ADAS-cog≦25) as severity indicators (Ritchie, C. W., Bush, A. I., Mackinnon, A., Macfarlane, S., Mastwyk, M., MacGregor, L., Kiers, L., Cherny, R., Li, Q.-X., Tammer, A., Carrington, D., Mavros, C., Volitakis, I., Xilinas, M., Ames, D., Davis, S., Beyreuther, K., Tanzi, R. E. & Masters, C. L. (2003) Metal-protein attenuation with iodochlorhydroxyquin (Clioquinol) targeting Aβ amyloid deposition and toxicity in Alzheimer disease: a pilot phase 2 clinical trial. Archives of Neurology, 60:1685-1691; Aisen, P. S., Saumier, D., Briand, R., Laurin, J., Gervais, F., Tremblay, P. & Garceau, D. (2006) A Phase Il study targeting amyloid-β with 3APS in mild-to-moderate Alzheimer disease. Neurology, 67:1757-1763). In the rember™ study, it was found that the MMSE≦19 subject group actually comprises a mix of 40% CDR-moderates with 60% CDR-milds. Likewise, the ADAS-cog>25 group comprises a mix of 41% CDR-moderates and 59% CDR-milds (see Tables 1 and 2). This means that the use of either MMSE≦19 or ADAS-cog>25 as an indicator of severity, as used conventionally in clinical trials of cognitively active drugs, has the effect of averaging the underlying difference between the milds and the moderates in a clinical trial setting. CDR severity was a much more powerful predictor of decline at 24 weeks than other baseline scores, and once CDR was included in the analysis, the other baseline scores became irrelevant.

Tables 1 and 2 show breakdown of randomised population by CDR severity vs. (a) MMSE severity grouping and (b) ADAS-cog severity grouping.

TABLE 1
Grouping by MMSE severity at baseline

| MMSE severity | CDR-mild | CDR-moderate | Total |
|---|---|---|---|
| (−Inf, 19] | 89 (60%) | 60 (40%) | 149 (100%) |
| (19, Inf] | 163 (94%) | 10 (6%) | 173 (100%) |

TABLE 2
Grouping by ADAS-Cog severity at baseline

| MMSE severity | CDR-mild | CDR-moderate | Total |
|---|---|---|---|
| (−Inf, 25] | 175 (91%) | 17 (9%) | 192 (100%) |
| (25, Inf] | 77 (59%) | 53 (41%) | 130 (100%) |

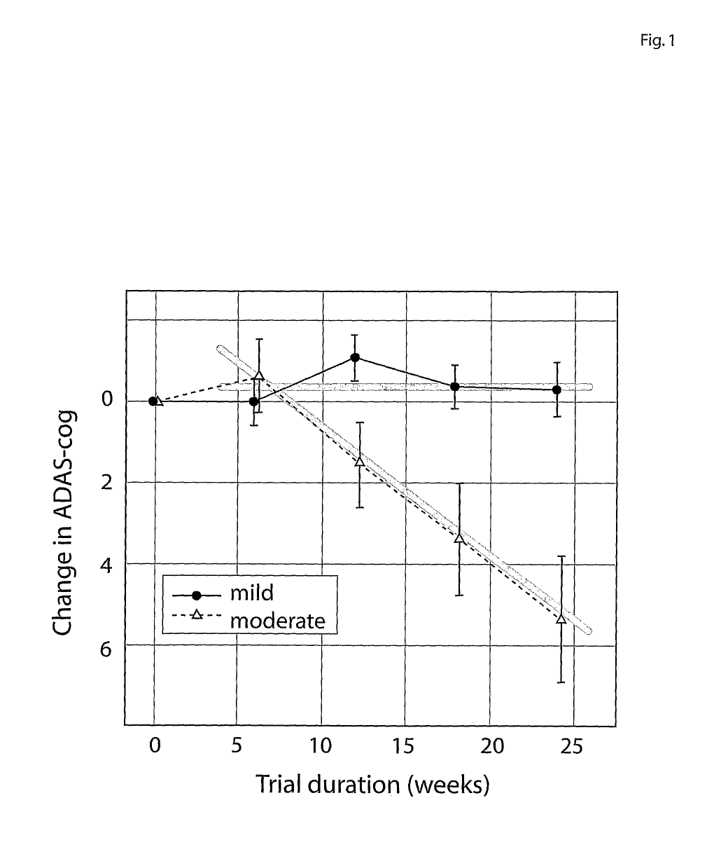

The use of CDR as a pre-specified stratification variable in rember™ study led to the discovery that there exists a clear dichotomy in disease progression based on CDR severity at baseline over 24 weeks. Subjects on placebo who were classified as CDR-mild did not decline over 24 weeks on either the ADAS-cog or MMSE scales (or indeed any of the non-cognitive scales), whereas subjects on placebo who were classified as CDR-moderate showed substantial decline. This is shown in FIG. 1 and Table 3.

TABLE 3
CDR-severity and placebo/comparator decline at 24 weeks⁽¹⁾

| ADAS-cog | | | | MMSE | | | |
|---|---|---|---|---|---|---|---|
| Baseline | Decline | 95% CI | p-value⁽²⁾ | Baseline | Decline | 95% CI | p-value⁽²⁾ |

CDR-mild placebo decline

| | | | | | | | |
|---|---|---|---|---|---|---|---|
| 21.72 | −0.56 | −1.89, 0.77 | 0.409 | 20.73 | −0.06 | −0.95, 0.83 | 0.890 |

CDR-moderate placebo decline

| | | | | | | | |
|---|---|---|---|---|---|---|---|
| 35.19 | +5.83 | 3.23, 8.44 | <0.0001 | 14.46 | −1.97 | −3.79, −0.15 | 0.0363 |

⁽¹⁾Based on two-slope linear mixed-effects model allowing change after 24 weeks.
⁽²⁾p-value is from a test of whether the estimated decline was different from zero.

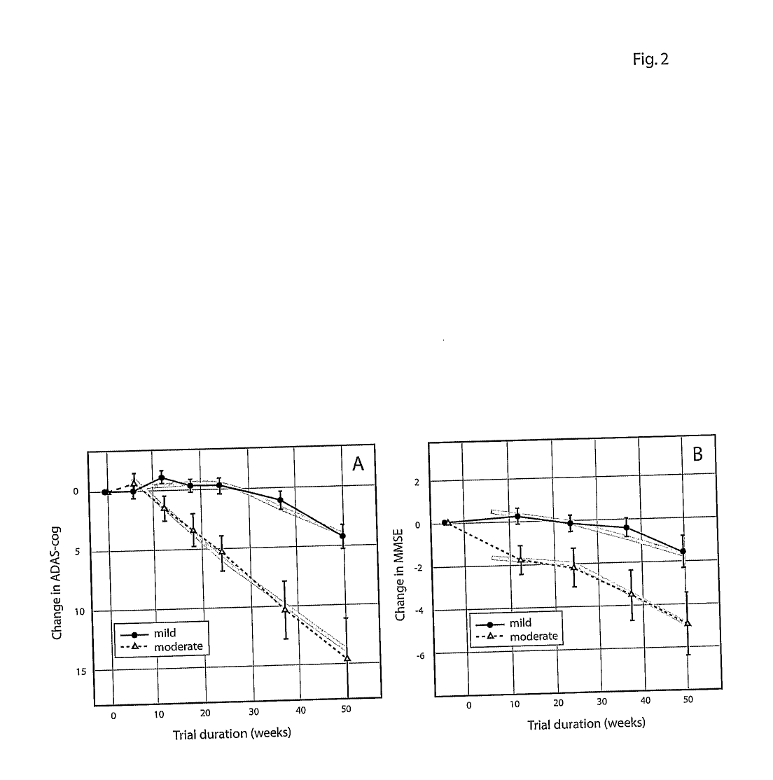

However, this non-decline was temporary. When observed over 50 weeks, CDR-mild subjects were seen to decline on both the ADAS-cog and MMSE scales, as shown in FIG. 2 and Table 4.

TABLE 4
CDR-severity and placebo/comparator decline at 50 weeks⁽¹⁾

| ADAS-cog | | | | MMSE | | | |
|---|---|---|---|---|---|---|---|
| Baseline | Decline | 95% CI | p-value⁽²⁾ | Baseline | Decline | 95% CI | p-value⁽²⁾ |

CDR-mild inferred placebo decline

| | | | | | | | |
|---|---|---|---|---|---|---|---|
| 21.72 | 4.00 | 2.40, 5.56 | <0.0001 | 20.73 | −1.70 | −2.68, −0.72 | 0.0010 |

CDR-moderate placebo decline

| | | | | | | | |
|---|---|---|---|---|---|---|---|
| 35.19 | 13.81 | 11.20, 16.42 | <0.0001 | 14.46 | −4.88 | −6.50, −3.33 | <0.0001 |

⁽¹⁾Based on two-slope linear mixed-effects model allowing change after 24 weeks.
⁽²⁾p-value is from a test of whether the estimated decline is different from zero.

CDR-moderates continued to decline over weeks 24 to 50 in the placebo-low treatment arm at a rate which was indistinguishable from the rate over weeks 0 to 24 (0.31 ADAS-cog units per week). By contrast, the non-decline of placebo subjects who were CDR-mild at baseline was temporary. Decline became evident over weeks 24 to 50. There was a highly significant change in slope at 24 weeks (p<0.0001). The rate of deterioration over weeks 24 to 50 was +0.17 ADAS-cog units per week, i.e. just over half the rate observed in CDR-moderate subjects. The difference in the rate of deterioration over weeks 24 to 50 between milds and moderates was also significant (p=0.0217).

Therefore, it is possible to detect decline in subjects who are CDR-mild at baseline, but it is necessary to extend the study for at least 50 weeks. Furthermore, it is necessary to separate the analysis of mild and moderate subjects. If an aggregate efficacy result is to be presented, it is necessary to account to the mix of mild and moderate subjects in the randomised population, as the estimated mean effect size for a disease-modifying treatment will be determined by the aggregate rate of placebo-decline in the placebo treatment arm.

Finally, given that a longer trial period is necessary to detect a treatment effect in CDR-mild AD, it is necessary to devise an imputation method to compensate for biased drop-out over longer trial durations. This last issue is discussed further below.

We first develop a better understanding of the basis of non-decline in mild AD as defined by CDR.

### Example 2

### Cognitive Reserve

Although there is a general relationship between increased load of brain pathology and decline in cognitive function, this relationship does not explain all of the variance in the data. Attempts to explain the variability have been formulated in terms of the concepts of “brain reserve” or “cognitive reserve”, which relate to two different theoretical formulations (Stern, Y. (2002) What is cognitive reserve? Theory and research application of the reserve concept. Journal of the International Neuropsychological Society, 8:448-460). The first is the passive brain reserve model, where reserve is defined by brain size or neuronal count. It is described as passive because it is defined in terms of the amount of damage or burden an individual can withstand before clinical symptoms appear. The second, or active cognitive reserve model, suggests that the brain can compensate for pathological burden by recruiting other processes to perform tasks compromised by disease (Stern, Y., Richards, M., Sano, M., Mayeux, R. (1993) Comparison of cognitive changes in patients with Alzheimer's and Parkinson's disease. Archives of Neurology, 50:1040-1045). Thus, according to the cognitive reserve hypothesis, individuals who have had greater amounts of reserve-enhancing experiences, such as education, are better able to cope with the brain damage or dysfunction brought about by aging and disease. It is difficult to measure cognitive reserve directly, but it has been suggested that education and occupation are proxies for this active adaptive capacity. These models are not mutually exclusive.

A study undertaken in Aberdeen (Scotland) has helped to resolve how cognitive reserve operates. The Aberdeen group have made use of a unique data base arising from Aberdeen birth cohorts of 1921 and 1936 (Whalley L. J., Deary, I. J. (2001) Longitudinal cohort study of childhood IQ and survival up to age 76. British Medical Journal, 322:819). These entire birth cohorts had formal IQ testing at age 11. As these individuals have reached the age of risk of dementia, they have been studied using repeated cognitive testing and brain imaging. Demographic and lifestyle measures have also been recorded, such as educational and occupational experience, with the aim of assessing the impact of reserve proxies on old age cognition. A cross-sectional study using estimates of brain pathology such as lesion count (Leaper, S. A., Murray, A. D., Lemmon, H. A., Staff, R. T., Deary, I. J., Crawford, J. R., Whalley, L. J. (2001) Neuropsychologic correlates of brain white matter lesions depicted on MR images: 1921 Aberdeen Birth Cohort. Radiology, 221:51-55) and brain volume (Staff, R. T., Murray, A. D., Deary, I. J., Whalley, L. J. (2006) Generality and specificity in cognitive aging: A volumetric brain analysis. NeuroImage, 30:1433-1440) has shown that over the life span, education and occupation protect individuals from cognitive decline in the face of pathological changes associated with aging (Staff, R. T., Murray, A. D., Deary, I. J., Whalley, L. J. (2004) What provides cerebral reserve? Brain, 127:1191-1199.).

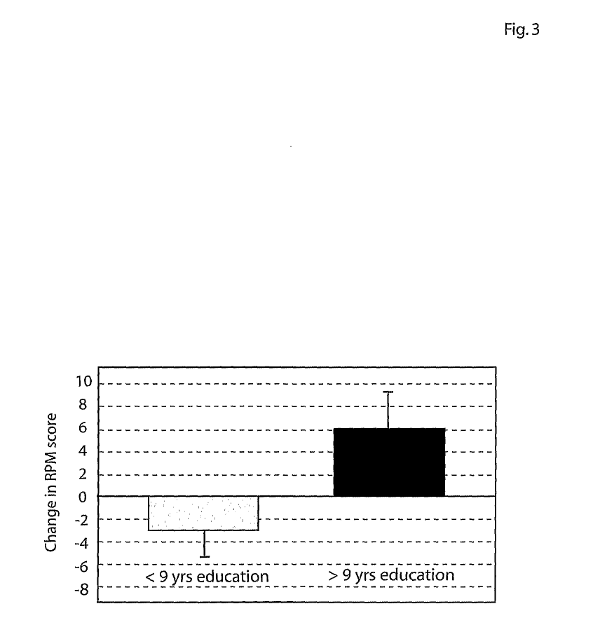

The Aberdeen group undertook a longitudinal study of old age pathological burden using volumetric analyses using MRI. This has shown that over one year, there was an average loss of 6.8 ml of grey matter and 1.8 ml of white matter among the cohorts. They postulated that this change is related to decline in cognitive performance, measured by particular reasoning and memory functions. However, they found that for the same loss of brain matter (burden), education protected against the expected cognitive deficit. They also found that while education was protective (p<0.003), IQ at age 11, occupation and total intra-cranial volume were not (Staff et al., manuscript in preparation). This is shown in FIG. 3. Change in cognitive functioning was measured over 1 year. After adjustment for premorbid intelligence (i.e., at age 11), loss of brain matter (burden) and gender, decline was seen only in subjects who had less than 9 years of schooling. There was improvement over 1 year in subjects who had more than 9 years of schooling, indicating that they had learned from the test presented one year earlier despite, having a degree of pathological burden which in less educated subjects led to decline in performance over one year.

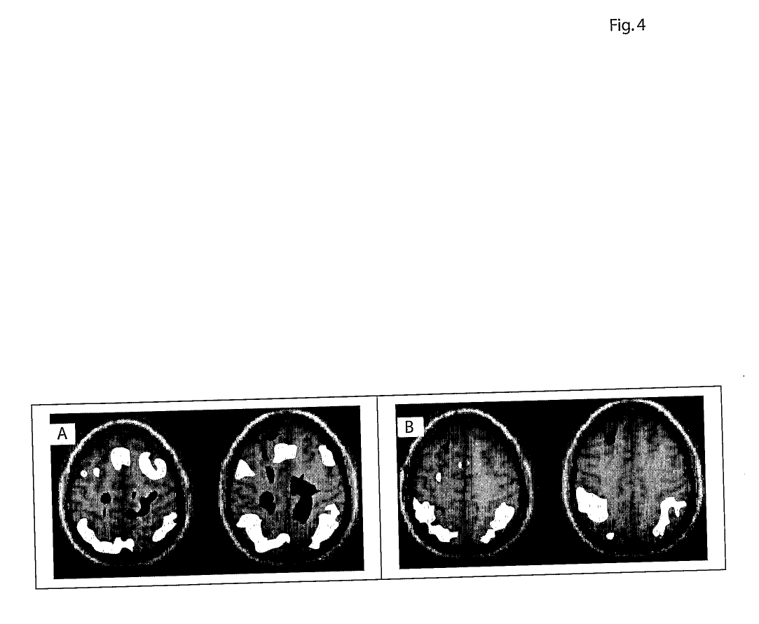

Further studies were undertaken to examine which regions of the brain were activated (increasing blood flow red) or deactivated (decreasing blood flow blue) to solve a working memory task in cognitive decliners vs. those who were aging “successfully” (i.e., without normal loss of brain matter), as shown in FIG. 4.

They found that the part of the brain performing the task was different in the declining group, indicating a difference in the functional pattern of brain activity used in the two groups in executing the same task. When proxies of reserve were considered, the results indicated that performance in the cognitive task was dependant on the degree of reserve (Waiter, G., Fox, H., Murray, A., Starr, J., Staff, R., Bourne, V., Whalley, L., Deary, I. J. (2007) Is retaining the youthful functional anatomy underlying speed of information processing a signature of successful cognitive aging?: an event related fMRI study of inspection time performance. NeuroImage, in press). It is this cognitive flexibility that appears to be the key factor which makes cognitive testing inaccurate with regard to measuring the effects of brain pathology.

In general, two stages of failure in a cognitive task can be envisaged from these studies: (1) task can still be performed, but requires compensation via additional effort or functional relocation; (2) outright task failure despite attempts at compensation. A recent study has identified brain regions in the frontal lobe that contribute to cognitive reserve (Stern, Y., Zarahn, E., Habeck, C., Holtzer, R., Rakitin, B. C., Kumar, A., Flynn, J., Steffener, J., Brown, T. (2007) A common neural network for cognitive reserve in verbal and object working memory in young but not old. Cerebral Cortex, in press). These results emphasize the need for early treatment of the disease to prevent the brain damage associated with early Braak stages and to preserve cognitive reserve, and not to delay treatment until symptoms of cognitive decline can be detected by standard clinical rating scales. Given that irreversible brain damage occurs in the medial temporal lobes well before the appearance of overt clinical decline (i.e. at Braak stage 2), these results emphasise the need to have improved diagnostic detection of early Braak stages (see e.g. WO02/075318), and the importance of prophylactic treatments aiming to prevent progression of neurofibrillary degeneration (see e.g. WO96/030766), even in the absence of clinical evidence of cognitive decline.

Although not having been appreciated in the art, and as discussed further below, cognitive reserve was found to be the primary factor responsible for the failure to detect to clinical decline in subjects who were CDR-mild at baseline, and who were randomised to placebo treatment. This was demonstrated by the surprising discovery that the same subjects receiving placebo who showed no evidence of cognitive decline in any of a broad range of psychometric tests used over 24 weeks, nevertheless showed prominent physiological decline over 6 months, amounting to a loss of 8% of functioning neuronal volume, as shown by decline in cerebral blood flow.

### Example 3

### Failure to Demonstrate Treatment Efficacy Over 24 Week Study by Psychometric Testing in CDR-Mild AD

Methods of Analysis

Two analysis methods are presented for each OC and LOCF analysis: linear least-squares and linear mixed-effects models of change in ADAS-cog score from baseline over 24 weeks. After finding that the baseline severity term was highly significant in all initial analyses, further analyses were conducted which included the treatment: severity:interaction term. Severity as defined by CDR was used as the baseline stratification variable. A further ANCOVA analysis of 24-week LOCF data was undertaken using only the analysis of change in ADAS-cog score at the 24-week assessment point using no-interaction and interaction models.

ITT/OC Analysis

No-Interaction Models

The treatment effect in the linear least squares and linear mixed-effects no-interaction models applied to the whole ITT/OC population demonstrated a statistically significant effect at the 60 mg dose in the linear least-squares model and a borderline significant effect in the more robust linear mixed effects model. The effect of baseline severity was highly significant.

The output of the models is given in Table 5.

TABLE 5a
ITT/OC analyses without interaction term

| | Linear least squares | | | Linear mixed effects | | |
|---|---|---|---|---|---|---|
| (in ADAS-cog units) | Estimate | 95% CI | p-value | Estimate | 95% CI | p-value |
| intercept⁽¹⁾ | 0.45 | −0.57, 1.47 | 0.387 | 0.10 | −1.46, 1.66 | 0.897 |
| low(100 mg)⁽²⁾ | 0.08 | −0.68, 0.83 | 0.845 | −0.04 | −1.20, 1.13 | 0.949 |
| 30 mg⁽²⁾ | 0.00 | −0.84, 0.83 | 0.997 | −0.07 | −1.38, 1.23 | 0.914 |
| 60 mg⁽²⁾ | −0.95 | −1.75, −0.15 | 0.0201 | −1.13 | −2.36, 0.11 | 0.0736 |
| moderate severity⁽²⁾ | 3.69 | 2.36, 5.03 | <0.0001 | 3.61 | 2.06, 5.17 | <0.0001 |

⁽¹⁾The intercept term is defined for mild, female, group 1 centres (Aberdeen & Birmingam), age >75 yrs, previously treated with AChE inhibitor or memantine, placebo, week 24; the p-value is from a test of whether the estimated value is significantly different from zero.
⁽²⁾The p-value is from a test of whether the estimated value is significantly different from the intercept term.

Interaction Models

When severity was included as an interaction term, the treatment effect of the 60 mg dose was found to be significant only in the group that was CDR-moderate at baseline. Prior treatment history, baseline ADAS-cog, and smaller centres remained as potentially significant cofactors.

The output of the model is given in Table 6.

TABLE 6
ITT/OC analyses with interaction term

| | Linear least squares | | | Linear mixed effects | | |
|---|---|---|---|---|---|---|
| (in ADAS-cog units) | Estimate | 95% CI | p-value | Estimate | 95% CI | p-value |
| intercept⁽¹⁾ | 0.54 | −0.50, 1.58 | 0.310 | 0.23 | −1.37, 1.82 | 0.7779 |
| mild: low(100 mg)⁽²⁾ | −0.05 | −0.88, 0.77 | 0.903 | −0.21 | −1.49, 1.06 | 0.741 |
| mild: 30 mg⁽²⁾ | 0.43 | −0.54, 1.41 | 0.385 | 0.33 | −1.18, 1.85 | 0.667 |
| mild: 60 mg⁽²⁾ | −0.66 | −1.53, 0.21 | 0.139 | −0.77 | −2.12, 0.57 | 0.259 |
| mod: low(100 mg)⁽²⁾ | 0.99 | −0.84, 2.82 | 0.288 | 1.13 | −1.67, 3.92 | 0.427 |
| mod: 30 mg⁽²⁾ | 1.11 | −2.71, 0.48 | 0.171 | −1.09 | −3.56, 1.38 | 0.386 |
| mod: 60 mg⁽²⁾ | −2.74 | −4.66, −0.83 | 0.0049 | −3.23 | −6.14, −0.32 | 0.0299 |
| moderate severity⁽²⁾ | 4.32 | 2.67, 5.97 | <0.0001 | 4.23 | 2.04, 6.42 | 0.0002 |

⁽¹⁾The intercept term is defined for mild, female, group 1 centres (Aberdeen & Birmingam), age >75 yrs, previously treated with AChE inhibitor or memantine, placebo, week 24; the p-value is from a test of whether the estimated value is significantly different from zero.
⁽²⁾The p-value is from a test of whether the estimated value is significantly different from the intercept term.

ITT/LOCF Analysis

No-Interaction Models

The treatment effect in the no-interaction models applied to the whole ITT/LOCF population demonstrated a statistically significant effect at the 60 mg dose. The effect of baseline severity was highly significant. Prior treatment history and smaller centres were also significant cofactors.

The output of the model is given in Table 7a.

TABLE 7a
ITT/LOCF analyses without interaction term

| | Linear least squares | | | Linear mixed effects | | |
|---|---|---|---|---|---|---|
| (in ADAS-cog units) | Estimate | 95% CI | p-value | Estimate | 95% CI | p-value |
| intercept⁽¹⁾ | 0.75 | −0.22, 1.72 | 0.128 | 0.29 | −1.25, 1.82 | 0.713 |
| low(100 mg)⁽²⁾ | −0.03 | −0.74, 0.68 | 0.934 | −0.03 | −1.21, 1.15 | 0.960 |
| 30 mg⁽²⁾ | 0.04 | −0.76, 0.84 | 0.919 | 0.04 | −1.29, 1.37 | 0.951 |
| 60 mg⁽²⁾ | −1.18 | −1.93, −0.43 | 0.0021 | −1.18 | −2.43, 0.06 | 0.0684 |
| moderate severity⁽²⁾ | 2.86 | 1.65, 4.07 | <0.0001 | 3.05 | 1.56, 4.54 | <0.0001 |

⁽¹⁾The intercept term is defined for mild, female, group 1 centres (Aberdeen & Birmingam), age >75 yrs, previously treated with AChE inhibitor or memantine, placebo, week 24; the p-value is from a test of whether the estimated value is significantly different from zero.
⁽²⁾The p-value is from a test of whether the estimated value is significantly different from the intercept term.

The treatment effect in the no-interaction models applied to the whole ITT/LOCF population failed to demonstrate a statistically significant effect at the 60 mg dose when a general linear model approach (LM or ANCOVA) was applied to only the 24-week assessment data, although the effect of baseline severity was again highly significant. No other cofactors were significant in this analysis. The output of the model is given in Table 7b.

TABLE 7b
ITT/LOCF LM analysis without interaction term

| (In ADAS-cog units) | Estimate | 95% CI | p-value⁽¹⁾ |
|---|---|---|---|
| low (100 mg) | −0.78 | −2.42, 0.84 | 0.343 |
| 30 mg | −0.04 | −1.87, 1.79 | 0.966 |
| 60 mg | −1.04 | −2.76, 0.68 | 0.235 |

⁽¹⁾The p-value is from a test of whether the value is significantly different from placebo.

Interaction Models

When the severity was included as an interaction term, the treatment effect of the 60 mg dose was found to be significant only in the group that was CDR-moderate at baseline.

The output of the model is given in Table 8a.

TABLE 8a
ITT/LOCF analyses with interaction term

| | Least squares | | | Mixed effects | | |
|---|---|---|---|---|---|---|
| (in ADAS-cog units) | Estimate | 95% CI | p-value | Estimate | 95% CI | p-value |
| intercept⁽¹⁾ | 0.88 | −0.11, 1.87 | 0.0808 | 0.42 | −1.15, 1.99 | 0.600 |
| mild: | −0.18 | −0.97, 0.60 | 0.642 | −0.18 | −1.48, 1.11 | 0.778 |
| low(100 mg)⁽²⁾ | | | | | | |
| mild: 30 mg⁽²⁾ | 0.46 | −0.47, 1.39 | 0.334 | 0.46 | −1.08, 2.00 | 0.558 |
| mild: 60 mg⁽²⁾ | −0.73 | −1.55, 0.09 | 0.0815 | −0.73 | −2.09, 0.63 | 0.291 |
| mod: | 1.06 | −0.64, 2.77 | 0.222 | 1.06 | −1.76, 3.88 | 0.459 |
| low(100 mg)⁽²⁾ | | | | | | |
| mod: 30 mg⁽²⁾ | −1.05 | −2.57, 0.47 | 0.175 | −1.05 | −3.56, 1.46 | 0.411 |
| mod: 60 mg⁽²⁾ | −3.70 | −5.46, −1.94 | <0.0001 | −3.70 | −6.61, −0.79 | 0.0130 |
| moderate severity⁽²⁾ | 3.63 | 2.08, 5.17 | <0.0001 | 3.81 | 1.63, 5.99 | 0.0007 |

⁽¹⁾The intercept term is defined for mild, female, group 1 centres (Aberdeen & Birmingam), age >75 yrs, previously treated with AChE inhibitor or memantine, placebo, week 24; the p-value is from a test of whether the estimated value is significantly different from zero.
⁽²⁾The p-value is from a test of whether the estimated value is significantly different from the intercept term.

When the severity was included as an interaction term in the LM/ANCOVA analysis of the ITT/LOCF data, the treatment effect of the 60 mg dose was likewise found to be significant only in the group that was CDR-moderate at baseline. The output of the model is given in Table 8b.

TABLE 8b
ITT/LOCF LM analysis with interaction term

| (In ADAS-cog units) | Estimate | 95% CI | p-value⁽¹⁾ |
|---|---|---|---|
| mild: | −0.79 | −2.51, 1.05 | 0.421 |
| low (100 mg)⁽²⁾ | | | |
| mild: 30 mg⁽²⁾ | 1.04 | −1.08, 3.17 | 0.335 |
| mild: 60 mg⁽²⁾ | −0.20 | −2.07, 1.68 | 0.838 |
| mod: | −0.40 | −4.29, 3.49 | 0.838 |
| low (100 mg)⁽²⁾ | | | |
| mod: 30 mg⁽²⁾ | −3.00 | −6.47, 0.47 | 0.090 |
| mod: 60 mg⁽²⁾ | −5.42 | −9.44, −1.40 | 0.0084 |

⁽¹⁾The p-value is from a test of whether the value is significantly different from placebo.

Subgroup Analysis—ADAS-cog in CDR Moderate Subjects

The primary outcome analysis provided a robust basis for subgroup analysis with particular attention to the CDR-moderate subgroup over 24 weeks. This section presents ITT/OC analyses of ADAS-cog over the first 24 weeks of treatment in subjects who were CDR moderate at baseline.

ITT/OC Analysis in CDR-Moderate Subjects at 24 Weeks

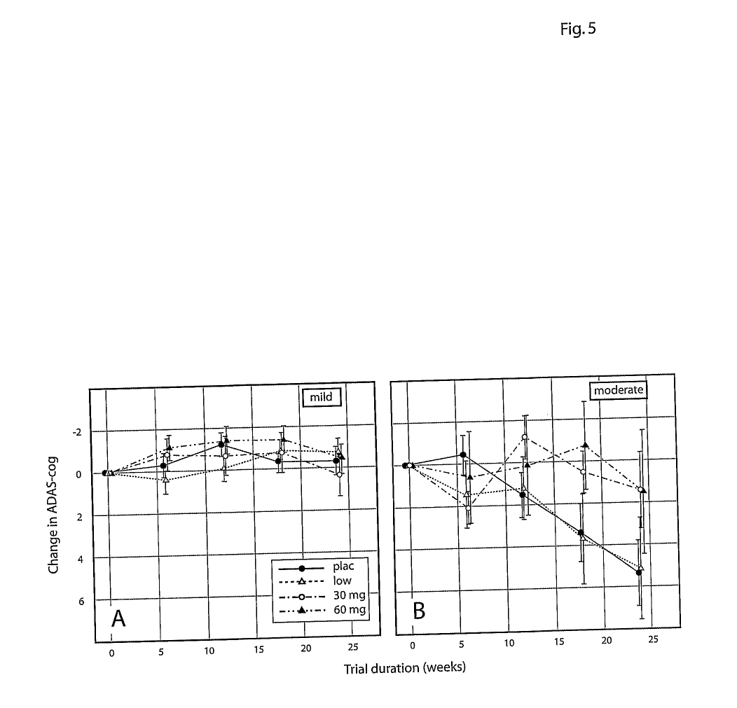

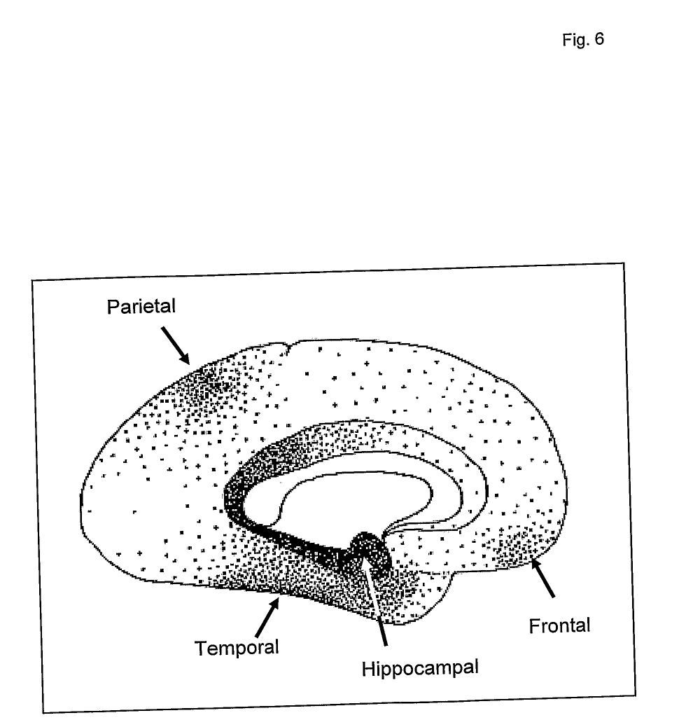

This analysis uses a mixed-effects model with a random per-patient coefficient and a fitted straight line response curve. The effect of rember™ on ADAS-cog score in CDR-moderate subjects after 24 weeks is shown in FIG. 5. In this chart the labelling conventions of “plac” refers to placebo, “low” refers to low (100 mg) dose tid, “30 mg” refers to 30 mg dose tid and “60 mg” refers to 60 mg dose tid. The shaded lines are best-fits calculated using the linear mixed effects random coefficients model. Table 9 shows overall change from week 0 to week 24, and Table 10 shows effect size at week 10.

TABLE 9
ADAS-cog change from baseline at 24 weeks in CDR-moderates

| (In ADAS-cog units) | Estimate | 95% CI | p-value⁽¹⁾ |
|---|---|---|---|
| placebo | 5.05 | 2.83, 7.27 | <0.0001 |
| low (100 mg) | 4.63 | 1.52, 7.74 | 0.0042 |
| 30 mg | 1.03 | −1.39, 3.45 | 0.398 |
| 60 mg | −0.36 | −3.57, 2.84 | 0.821 |

⁽¹⁾The p-value is from a test of whether the value is significantly different from zero

TABLE 10
ADAS-cog effect size at 24 weeks in CDR-moderates

| (In ADAS-cog units) | Estimate | 95% CI | p-value⁽¹⁾ |
|---|---|---|---|
| low (100 mg) | −0.42 | −4.24, 3.40 | 0.826 |
| 30 mg | −4.02 | −7.30, −0.74 | 0.0172 |
| 60 mg | −5.41 | −9.31, −1.52 | 0.0073 |

⁽¹⁾The p-value is from a test of whether the value is significantly different from placebo.

Conclusion from 24-Week Analyses of ITT Population

It is concluded that rember™ at 60 mg tid has efficacy in the entire ITT population of mild and moderate AD in both the OC and LOCF analyses, although the effect size was substantially underestimated (in the range −1.0 to −1.2 ADAS-cog units) in these populations because of pooling of CDR-mild and CDR-moderate subjects. In the no-interaction analyses, the effect of the 60 mg dose achieved statistical significance by the least-squares method and borderline statistical significance by the more conservative mixed effects method. CDR-severity at baseline was a highly significant cofactor in all analyses. When this cofactor was included in the ITT/OC and ITT/LOCF analyses, the effect of rember™ was found to be significant at 24 weeks only in the subjects who were CDR-moderate at baseline. This was true for both mixed effects models of all data over 24 weeks, and linear modeling of only the data from the 24-week time-point. The mean effect sizes in moderate subjects were in the range −2.7 to −3.7 ADAS-cog units over 24 weeks in the analyses conducted in the entire ITT/OC population after in inclusion of severity as an interaction term in the analysis. In the linear model at the 24-week time-point, the effect size was −5.4 ADAS-cog units. There were no significant differences between the OC and LOCF analyses in the mixed effects analyses. It is concluded that the prespecified primary outcome analysis in the whole ITT population confirms the efficacy of rember™ at 24 weeks, and that it is appropriate to analyse CDR-mild and CDR-moderate subjects as separate subgroups in further analyses.

In the CDR-moderate subgroup of the ITT population, there was a decline of 5.1 ADAS-cog units over 24 weeks in patients treated with placebo. There was a similar decline of 4.6 ADAS-cog units in patients treated with the low (100 mg) dose, confirming minimal therapeutic efficacy of these capsules over 24 weeks due to problems with the formulation of this capsule. This provides a basis for consideration of the low (100 mg) treatment arm as equivalent to placebo when administered over 6 months for the purpose of analysing psychometric change. Thus, subjects originally receiving placebo over the first 24 weeks who were then switched to the low (100 mg) dose over weeks 24 to 50 could be considered to represent a suitable Least Exposed Dose comparator arm which was approximately equivalent to placebo over 50 weeks for the purpose of the ADAS-cog analysis, as for example in Table 4 and FIG. 5 above.

By contrast, no significant decline was seen at 24 weeks in CDR-moderate patients treated with rember™ at either the 30 mg or 60 mg doses. This translates into effect sizes of −4.0 and −5.4 ADAS-cog units at 24 weeks in CDR-moderate patients treated with 30 mg and 60 mg tid respectively. It is concluded that disease progression was arrested at 24 weeks in CDR-moderate patients by treatment with rember™ at either 30 mg tid or 60 mg tid.

It appears inherently implausible that treatment with rember™ should have therapeutic efficacy in CDR-moderate AD, a more advanced and rapidly progressing stage of the disease, and yet have no apparent efficacy in CDR-mild AD. In light of the apparent non-decline of CDR-mild subjects over 24 weeks on psychometric measures, it is concluded that this phenomenon substantially interferes with detection of therapeutic efficacy of disease-modifying treatments. This raises the problem of how to demonstrate disease-modifying efficacy in circumstances such as early stages of clinical AD (eg CDR-mild AD or MCI), or even earlier stages when clinical decline is not apparent. For example, Park et al. (2007) (Park, K. W., Pavlik, V. N., Rountree, S. D., Darby, E. J., Doody, R. S. (2007) Is functional decline necessary for a diagnosis of Alzheimer's disease? Dementia and Geriatric Cognitive Disorders, 24:375-379) recently compared two groups over 1 year: those with evidence of functional decline in activities of daily living (ADL) and those without. They found an essentially identical course irrespective of presence or absence of functional decline. They conclude that the application of current diagnostic criteria (such as DSM-IV criteria, which require evidence of functional decline in order to diagnose AD) has the effect of delaying diagnosis in approximately 15% of AD cases, and in more than half of these, the delay in diagnosis would be more than 1 year. With the advent of treatments such as rember™ which can prevent the destructive effects of the process of neurofibrillary degeneration (see below), particularly in medial temporal lobe structures, this delay in diagnosis appears to be both unwarranted and undesirable.

### Example 4

### Functional Brain Scan Elucidation of Mechanism Responsible for Apparent Non-Decline in CDR-Mild AD

In order to determine the basis of non-decline in CDR-mild AD subjects, an analysis of changes in functional brain scan was undertaken.

The rember™ study used SPECT with the ligand ^{99m}Tc-HMPAO or FDG PET at baseline to confirm diagnosis in certain study centres where this capability was available. Where possible, SPECT functional brain imaging was also used as a secondary outcome measure, comparing changes between baseline and visit 4 (18 weeks) as a response to treatment with rember™.

Study Design

Functional brain scans were included in the trial, both as a baseline stratification variable, and as a surrogate efficacy marker. There were 138 subjects in the SPECT cohort who had images both at baseline and at visit 4 (18 weeks). An ITT/OC analysis was conducted in all subjects with paired SPECT scans who were ongoing with medication at the time of the second scan (125). A subgroup of particular interest were subjects who were CDR-mild at baseline (100).

Subjects had their first scan at approximately the time of randomisation. Allowance was made in the case of newly diagnosed subjects who may have had a recent scan up to 3 months prior to randomisation, and these were not required to undergo a second baseline scan. In some centres a baseline scan was allowed after initiation of treatment. The mean inter-scan interval was 6 months (±1.2, sd). The range of inter-scan intervals was 4-11 months.

Scans were all sent to Aberdeen and were assessed by two independent nuclear medicine experts at the Aberdeen Royal Infirmary who were blinded as to treatment group and clinical information.

Patient Disposition and Characteristics

Functional brain scan data acquired in the rember™ trial were as listed in Table 11.

TABLE 11
HMPAO-SPECT scan data

| | | Number of | Number of subjects |
|---|---|---|---|
| | Number of | subjects | imaged twice and |
| | subjects | image | rejected due to |
| Imaging site | imaged | twice | poor quality |
| Aberdeen (AS, AN, CS, | 132 | 84 | 0 |
| BF⁽¹⁾) | | | |
| Bradford (BF) | 9 | 6 | 0 |
| Birmingham QEII (BH) | 8 | 8 | 0 |
| Blackpool (BP) | 6 | 4 | 0 |
| Glasgow (CM, CP) | 41 | 17 | 1 |
| Guildford (GF | 16 | 10 | 0 |
| Ipswich (IS) | 3 | 1 | 0 |
| Plymouth (PM) | 8 | 7 | 1 |
| Birmingham City (SW) | 4 | 1 | 1 |
| Total | 227 | 138 | 3 |

⁽¹⁾Because of inadequate scanning facilities at Bradford, some subjects were brought to Aberdeen for scanning.

Population characteristics of ITT/OC subjects with two scans are shown in Table 12.

TABLE 12
ITT/OC population with two analysable scans

| | | | | | | | Days | | | Previous | | | |
|---|---|---|---|---|---|---|---|---|---|---|---|---|---|
| | | AD PATH | | VASC PATH | | | V1-V4 | Sex | | treatment | | Baseline severity | |
| | Total | AD | No AD | VASC | No VASC | Age (SD) | (SD) | Female | Male | NO | YES | Mild | Moderate |
| low(100 mg) | 33 | 29 | 4 | 10 | 23 | 69.6 (9.6) | 184.0 (36.4) | 15 | 18 | 24 | 9 | 26 | 7 |
| 60 mg | 30 | 26 | 4 | 11 | 19 | 69.2 (10.9) | 197.1 (56.3) | 20 | 10 | 21 | 9 | 24 | 6 |
| 30 mg | 12 | 12 | 0 | 5 | 7 | 70.7 (8.4) | 175.7 (30.2) | 3 | 9 | 10 | 2 | 8 | 4 |
| placebo | 50 | 43 | 7 | 14 | 36 | 74.6 (7.6) | 189.3 (30.2) | 22 | 28 | 39 | 11 | 42 | 8 |
| | 125 | 110 | 15 | 40 | 85 | | | 60 | 65 | 94 | 31 | 100 | 25 |

Methodology

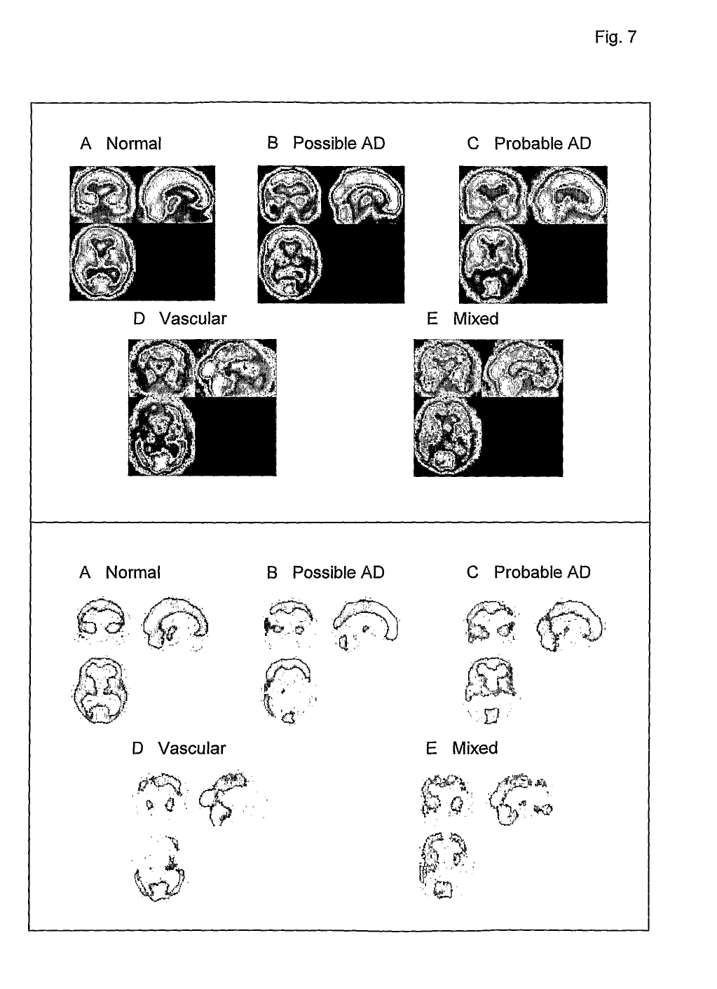

FIG. 7 illustrates typical SPECT scan appearances used to determine baseline functional scan diagnosis in some centres in addition to NINCDS-ADRDA and DSM IV clinical criteria.

Two analysis methods were used as outcome methods in the study: Region of Interest (ROI) Analysis and Statistical parametric (SPM) analysis

Each of these techniques has its advantages and disadvantages. The ROI approach is relatively simple to follow and gives an estimate for the blood flow at a particular location which can be tested with standard statistical methods. However, the ROI approach requires investigators to make assumptions about the location and volumetric extent of differences. Conversely, the SPM approach allows the investigator to test all locations and size or volumetric extent combinations, making it a more robust analytical tool. The disadvantage is that SPM is more difficult to implement and execute and is a technique restricted to expert centres.

ROI Analysis

The first analysis uses standardised brain regions based on the West Forest University (NC, U.S.A.) image analysis tool (“WFU-Pickatlas”) (Maldjian, J. A., Laurienti, P. J., Burdette, J. B., Kraft, R. A. (2003) An automated method for neuroanatomic and cytoarchitectonic atlas-based interrogation of fMRI data sets. NeuroImage, 19:1233-1239; Maldjian, J. A., Laurienti, P. J., Burdette, J. H. (2004) Precentral gyrus discrepancy in electronic versions of the Talairach Atlas. NeuroImage, 21:450-455). A standardised ROI was created for each lobe of the brain and divided into hemispheres for frontal, parietal, temporal and occipital lobes (8 ROIs), and cerebellum. These regions are illustrated below in FIG. 8. Counts derived from each of the first eight regions were normalised with respect to counts in cerebellum to make allowance for inter-individual variation, and non-specific pharmacological effects of rember™. The cerebellum is minimally affected by AD-pathology.

For the ROI analysis, a single per-subject parameter was defined from 8 regional measurements per subject. This was calculated from a principal components analysis consisting of two factors: a general factor common to all lobes and a specific factor that explains the additional lobe-specific variance. The general factor value (GFV) per-subject used for further analysis was normalized to a value of 1 with a standard deviation of 0.15.

SPM Analysis

For the SPM analysis, software developed at University College London (UCL), Queens Square London was used. Images were spatially registered to a standard imaging template and smoothed. Parametric statistical models are assumed at each voxel, using a general linear model to define the data in terms of experimental and confounding (i.e., cofactor) effects, and residual variability. Further details can be found at the web site (http://www.fil.ion.ucl.ac.uk/spm/).

Correlation Between Baseline ADAS-cog Severity and Baseline Cerebral Blood Flow

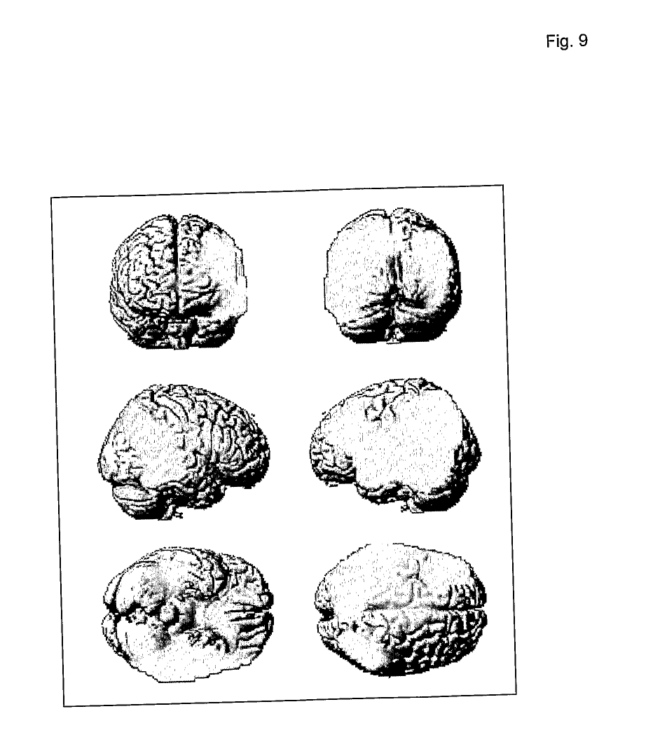

In the rember™ study, there was a strong correlation between baseline clinical severity as measured by ADAS-cog score and pathological burden as measured by baseline Regional Cerebral Blood Flow (rCBF). This was seen particularly in the left frontal, parietal and occipital lobes, as shown in FIG. 9. This is consistent with previous reports (Nebu, A., Ikeda, M., Fukuhara, R., et al. (2001) Relationship between blood flow kinetics and severity of Alzheimer's disease: Assessment of severity using a questionnaire-type examination, Alzheimer's disease assessment scale, cognitive sub-scale (ADAS(cog)). Dementia, Geriatric and Cognitive Disorders, 12:318-325), as is the asymmetric predominance of left-sided change (Kovalev, V. A., Thurfjell, L., Lundqvist, R., Pagani, M. (2006) Asymmetry of SPECT perfusion image patterns as a diagnostic feature for Alzheimer's disease. Medical image computing and computer-assisted intervention: MICCAI International Conference on Medical Image Computing and Computer-Assisted Intervention, 9:421-428).

There was also a significant association between age and baseline rCBF in the parietal and temporal lobes, confirming previous findings of Kemp et al., (Kemp, P. M., Holmes, C., Hoffmann, S. M., Bolt, L., Holmes, R., Rowden, J., Fleming, J. S. (2003) Alzheimer's disease: differences in technetium-99m HMPAO SPECT scan findings between early onset and late onset dementia. Journal of Neurology, Neurosurgery and Psychiatry, 74:715-719). Interval between scans also had a significant effect on between-scan change in cerebral blood flow. Other cofactors (sex and baseline clinical severity) were variable. History of previous treatment with AD-labelled drugs was also a significant cofactor, indicating that subjects who had elected to withdraw from prior treatment with AD-labelled drugs (predominantly AChE inhibitors) had more advanced disease.

Analysis of Changes in Functional Brain Scan in CDR-Mild Subjects Over Initial 6-Months of Rember™ Study

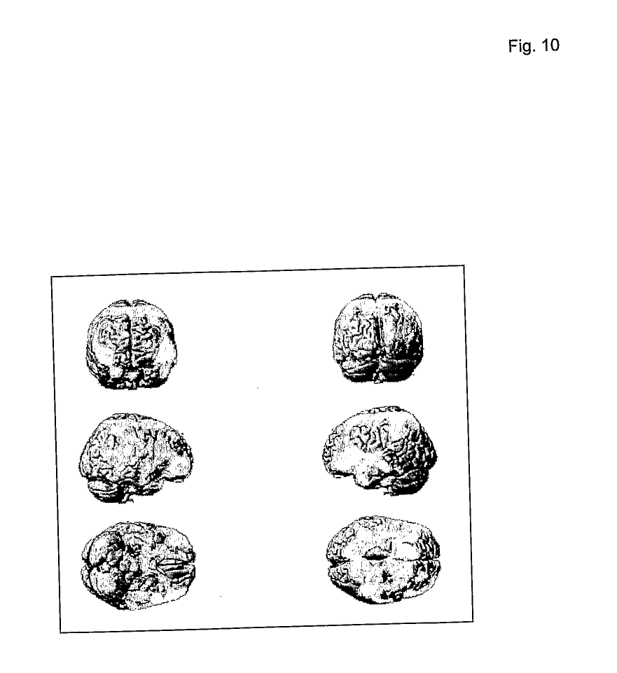

An analysis was conducted in the ITT/OC subgroup that was CDR-mild at baseline. This was of particular interest in light of the failure of the CDR-mild group to decline on ADAS-cog over of the first 24 weeks of the trial. FIG. 10 shows locations of regions of significant decline between baseline and visit 4 in subjects who were CDR-mild at baseline.

There was significant decline in functioning neuronal volume as shown by cerebral blood flow in subjects who were CDR-mild at baseline despite lack of evidence of decline on the ADAS-cog, the MMSE scale or any other psychometric scale.

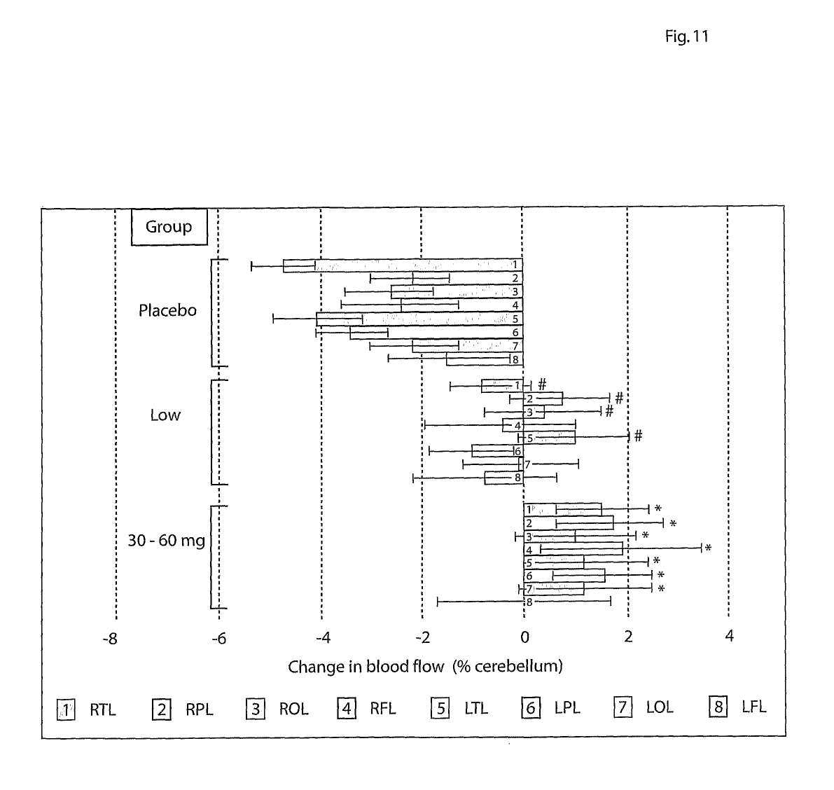

The extent of this decline and the effects of treatment with rember™ can be seen in FIG. 11. It is important to note that rember™ produced significant improvements in CDR-mild subjects at the 30/60 mg doses after 4 months of treatment. This improvement (ie difference from zero), can be seen in the mean general factor values in FIG. 11.

In subjects who were CDR-mild at baseline, the mean decline over 6 months in the general perfusion factor in subjects treated with placebo was −8.23% (95% confidence interval, [−11.89, −4.57]; p-value, <0.001). All lobes were found to decline significantly in subjects receiving placebo, the greatest declines being in the right and left temporal lobes. This decline did not occur in subjects receiving rember™ at 30/60 mg doses, and indeed there was overall evidence of improvement, which did not achieve statistical significance, although the difference in the group with respect to placebo was highly significant in most brain regions.

Treatment with rember™ at low (100) mg tid produced significant benefits with respect to placebo in right temporal lobe, right parietal lobe, right occipital lobe and left temporal lobe. Treatment with rember™ at 30/60 mg produced greater significant benefits with respect to placebo in the same regions (right temporal lobe, right parietal lobe, right occipital lobe and left temporal lobe), and also more widespread improvements affecting right frontal lobe, left parietal lobe, left occipital lobe.

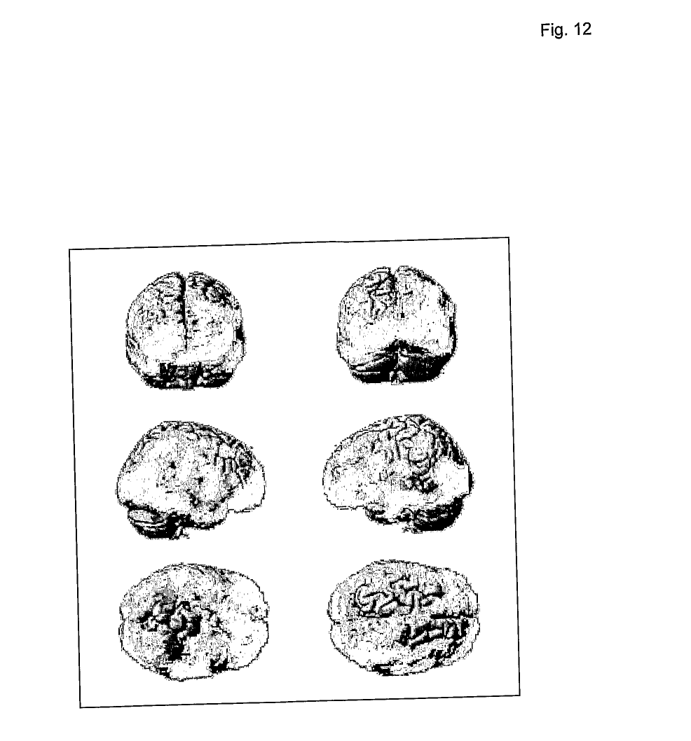

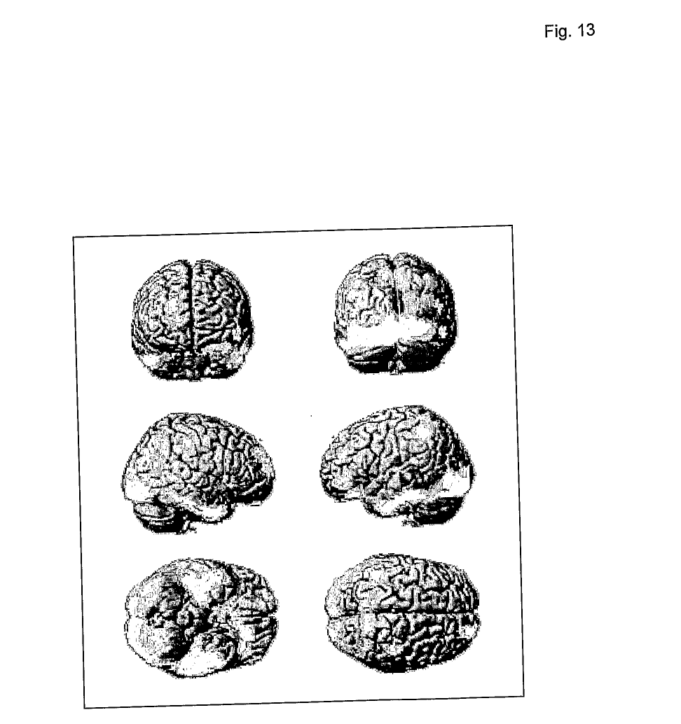

The treatment effects of rember™ in the CDR-mild group can be seen in the SPM analyses comparing placebo with 30/60 mg tid treatment (FIG. 12) and comparing placebo with treatment at the low (100 mg) dose (FIG. 13). As can be seen the extent of functional change is less at the low (100 mg) dose than at the 30/60 mg doses.

ITT/OC Change in rCBF Correlation with Change in ADAS-cog Score

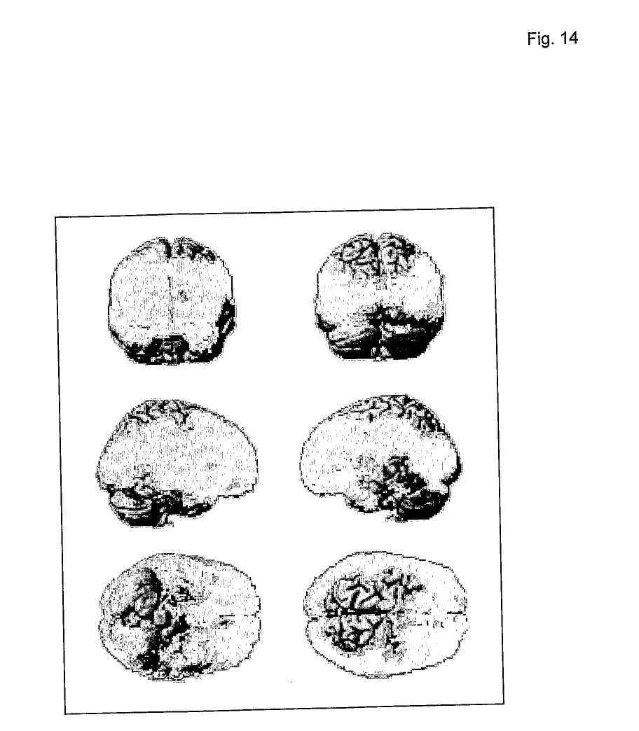

The final analysis examines whether SPECT scan could be used as a surrogate marker for clinical response. There was a strong correlation between change on SPECT scan and change as measured by ADAS-cog over 24 weeks in ITT/OC population treated with rember™ in FIG. 14.

All regions, other than right parietal lobe, showed positive correlation between ADAS-cog improvement and rCBF improvement. The region of highest correlation was the right temporal lobe (r=0.44, p<0.001). The general factor (GFV) also showed a highly significant correlation in the corresponding ROI analysis (r=0.46, p<0.001).

Reversal of Medial Temporal Lobe Pathology by Treatment with Rember™

As part of the rember™ study, there were 20 cases available where the subjects had been imaged before and after treatment with rember™ for 18 weeks using FDG PET. PET, as available in the rember™ study, had higher resolution than SPECT, and so permitted better definition of anatomical change in medial temporal lobe structures. It has been possible to demonstrate that rember has the capacity to reverse the characteristic pathology of medial temporal lobe. This is illustrated below in a case treated with rember™ at 60 mg tid for 18 weeks.

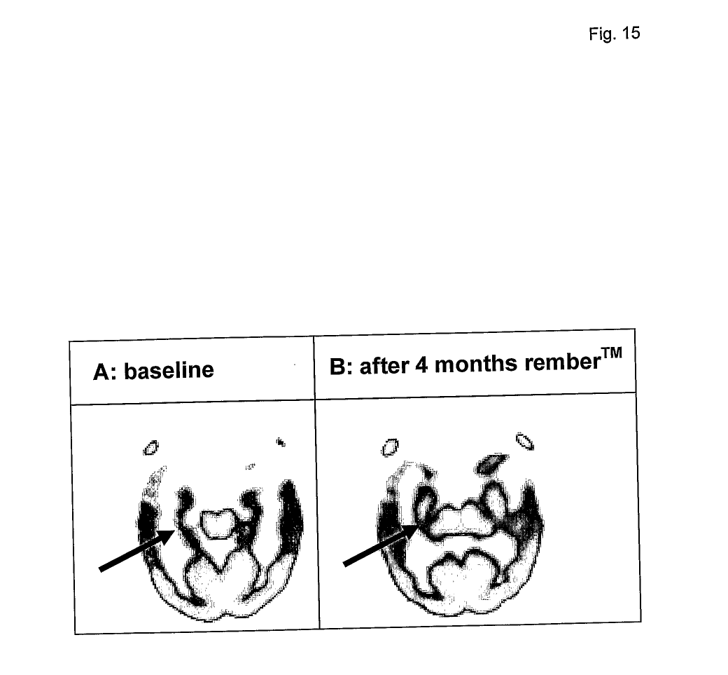

FIG. 15 shows the conversion of a subject with clear AD features (reduced perfusion in hippocampal (HC) and entorhinal cortex (ERC) regions) to normal scan features following treatment. This image is particularly striking as these are the regions affected by Tau pathology earliest and most severely in AD (Braak & Braak, 1991; Gertz et al., 1998; García-Sierra, F., Wischik, C. M., Harrington, C. R., Luna-Mu\check{n}oz, J. & Mena, R. (2001) Accumulation of C-terminally truncated tau protein associated with vulnerability of the perforant pathway in early stages of neurofibrillary pathology in Alzheimer's disease. Journal of Chemical Neuroanatomy, 22:65-77), and reversal of this kind is never normally seen in sequential scans in clinical populations.

The significance of this result for understanding the potential prophylactic benefit of treatment of the rember type can be better understood with reference to Example 7 below.

Additional Comments on PET Functional Brain Imaging

FDG-PET (Fluoro-Deoxy Glucose Positron Emission Tomography) and HMPAO-SPECT (Hexamethyl-Propylene-Amine-Oxime Single Photon Emission Tomography) provide complementary approaches to measuring functional changes in brain. In the study SPECT or PET functional brain imaging was conducted at baseline to confirm diagnosis in those centres where this capability was available. Where possible, SPECT and PET functional brain imaging was also used as a secondary outcome measure, comparing changes between baseline and visit 4 (18 weeks) as a response to treatment with rember™.

When used diagnostically, SPECT and PET both reveal a characteristic bilateral temporo-parietal defect. However, the underlying biological mechanisms of action of these imaging modalities differ. SPECT reports a cerebral blood flow image obtained ‘first pass’ after intravenous injection. PET reports an image of glucose uptake over a period of 3 hours after injection. Both report neuronal function in different ways. SPECT depends on local blood flow over a short time-course, and so provides an indirect measure of neuronal function, since neuronal oxygen demand is closely linked to cerebral blood flow. PET measures metabolic function more directly, but integrates glucose uptake over a longer time-course.

Analysis of the data from 19 subjects who had been imaged twice by PET was performed. These were made up of the following groups: placebo (n=7); 100 mg dose (n=4); and 60 mg dose (n=8). A substantial increase in glucose uptake in hippocampus and entorhinal cortex was observed in a case following treatment with rember™ at 60 mg tds (FIG. 15). The SPM analysis of the PET data now permits this observation to be generalised. Because of the small number of cases, the main results are presented for pooled rember™-treated cases (i.e. pooling 100 mg and 60 mg tid dose groups).

In contrast to the SPECT findings, decline in glucose uptake did not reach statistical significance in any brain region in placebo-treated subjects.

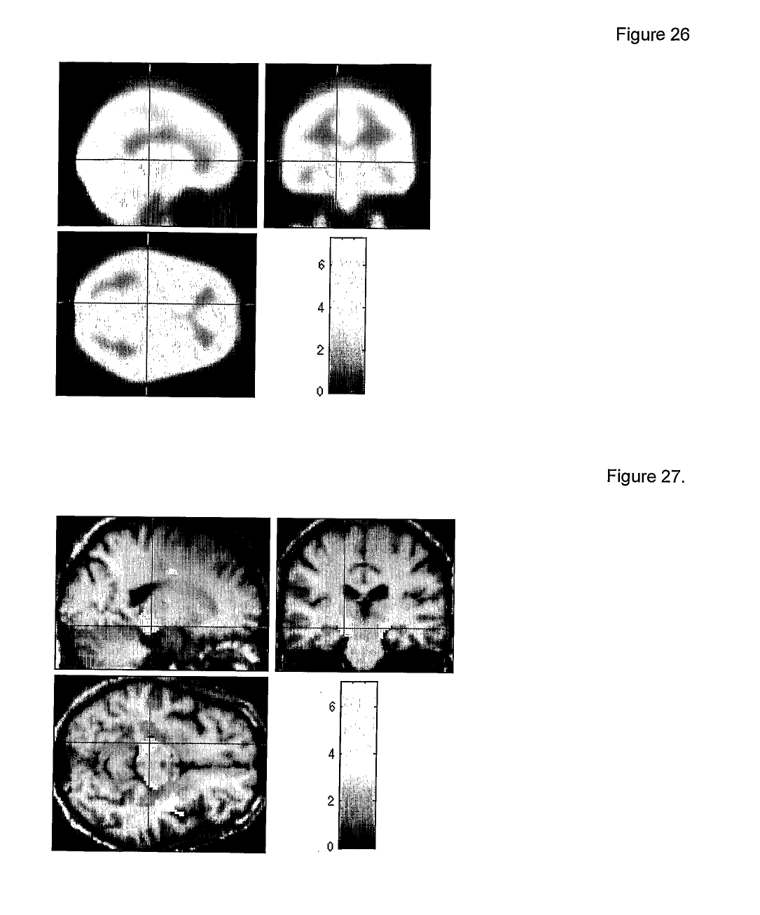

However, in rember™-treated subjects, there was a region of significant increase glucose uptake from the baseline image to the second PET scan at visit 4 (18 weeks) (correcting for multiple comparisons across the whole head). This was located in the left medial temporal lobe (MTL; hippocampus and entorhinal cortex) as shown in FIG. 26. This is a t-score map, with corresponding t-scores shown on the scale. When the data were re-analysed, making the assumption that changes were expected only in the medial temporal lobe (i.e. a small volume correction for multiple comparisons), the increase in FDG uptake was significant in the MTL structures bilaterally. Similar results were found when the 60 mg tid group group was examined separately.

Finally, there was a significant difference in glucose uptake with respect to placebo in subjects treated with rember™ at 60 mg or 100 mg tid. This is shown in FIG. 27. In this case, regions of difference are shown superimposed on an MRI-scan image of one of the subjects to show approximate locations of regions of significant difference.

The most striking feature of the PET results is that in subjects treated with rember™ at 60 mg or 100 mg tid there is evidence of increase in glucose uptake from baseline in the brain regions affected earliest and most severely in the progression of AD. The MTL structures, hippocampus and entorhinal cortex, are the regions of highest density of Tau aggregation pathology. The finding that treatment with rember™ produces an increase in glucose uptake selectively in these regions provides strong evidence supporting the mechanism of action of rember™ as a Tau aggregation inhibitor. That is, other possible non-specific mechanisms of action, such as general enhancement of mitochondrial metabolism, appear unlikely given the circumscribed regional selectivity of the effect.

There was concern in the design of the clinical trial that at more advanced stages of AD the damage to MTL structures would have become irreversible. Extracellular tangle counts increase early in entorhinal cortex and hippocampus, whereas there is almost no tangle-mediated neuronal destruction in neocortex until very late in the disease (Mukaetova-Ladinska et al., 2000; Garcia-Sierra et al., 2000). It was for this reason that stratification by baseline severity was prespecified as an important covariate in the Satistical Analysis Plan. The concern, based on the earlier post-mortem studies, was that more advanced disease would be less responsive to treatment with a Tau aggregation inhibitor. In the event, the study showed that the effect size of treatment with rember™ was larger in CDR-moderate than in CDR-mild AD, probably because the rate of placebo-decline is greater in CDR-moderate AD.

The PET data show that treatment with rember™ exerts its strongest metabolic effect in the medial temporal lobe structures. The dissolution of Tau aggregates produces a marked increase in functional activity as measured by enhancement in glucose uptake in the regions where the Tau pathology is most severe. The fact that a statistically significant effect could be demonstrated with such a small number of cases indicates that the effect size is large relative to the inherent variability of the data, and leads to the expectation that the effect is robust and will be readily demonstrable in larger case series.

The second striking feature of the present data is the apparent difference in the pattern of change produced by rember™ as seen by PET relative to that seen by SPECT. Although SPECT scans have a lower resolution, it may be possible to determine if there are corresponding MTL blood flow changes by altering the planes in which image reconstruction and registration are undertaken. As discussed further below, the two imaging modalities may not provide the same results as they are dependent on different mechanisms of action of the underlying imaging agents.

Whereas neuropathological studies have established the importance of the stereotyped regional hierarchy in the progression of Tau aggregation pathology in AD, β-amyloid pathology does not show this neuroanatomical specificity (Braak and Braak, 1991). The early role of the medial temporal lobe structures has been confirmed in longitudinal MRI studies. In cases of Mild Cognitive Impairment (MCI) atrophy in the hippocampus (Visser, P J, Scheltens, P, Verhey, F R J, Schmand, B, Launer, L J et al. (1999) Medial temporal lobe atrophy and memory dysfunction as predictors for dementia in subjects with mild cognitive impairment. J. Neurol. 246:477-485; Jack, C R, Jr., Petersen, R C, Xu, Y C, O'Brien, P C, Smith, G E et al. (1999) Prediction of AD with MRI-based hippocampal volume in mild cognitive impairment. Neurology 52:1397-1403; Mungas, D, Reed, B R, Jagust, W J, DeCarli, C, Mack, W J et al. (2002) Volumetric MRI predicts rate of cognitive decline related to AD and cerebrovascular disease. Neurology 59:867-873), and more particularly the entorhinal cortex (Stoub, T R, Bulgakova, M, Leurgans, S, Bennett, D A, Fleischman, D et al. (2005) MRI predictors of risk of incident Alzheimer disease: A longitudinal study. Neurology 64:1520-1524), are predictive of progression to AD.

The relationships between regional loss of grey matter (as measured by MRI), loss of perfusion (as measured by SPECT) and loss of cognitive function in specific domains are complex. There is a strong correlation between cognitive decline and decline in cerebral blood flow, particularly for the frontal lobes and less for the temporal lobes (Brown, D R P, Hunter, R, Wyper, D J, Patterson, J, Kelly, R C et al. (1996) Longitudinal changes in cognitive function and regional cerebral function in Alzheimer's disease: A SPECT blood flow study. J. Psychiatr. Res. 30:109-126 and confirmed in the clinical trial). Furthermore, it is generally recognised that there is posterior to anterior spread of perfusion defects with advancing disease (Matsuda, H, Kitayama, N, Ohnishi, T, Asada, T, Nakano, S et al. (2002) Longitudinal evaluation of both morphologic and functional changes in the same individuals with Alzheimer's disease. J. Nuc. Med. 43:304-311).

However there is not a simplistic relationship between regions of reduced perfusion and regions of loss of specific cognitive functions traditionally localised to those brain regions. This is because of the hierarchical nature of progression of pathology in AD: spread to the frontal lobes implies increased severity of pathology in brain regions affected earlier. Therefore, decline in frontal perfusion is also a marker of more global decline. Furthermore, there is not a simplistic relationship between regions of atrophy, measured by MRI, and SPECT perfusion defects. Thus, in affected areas, there is generally a greater reduction in volume than reduction in cerebral blood flow, and indeed there can be reductions in volume without any corresponding loss of cerebral blood flow (e.g. in hippocampus) in MCI/mild AD (Ibanez, V, Pietrini, P, Alexander, G E, et al. (1998) Regional glucose metabolic abnormalities are not the result of atrophy in Alzheimer's disease. Neurology 50:1585-1593). Matsuda et al. (2002) found in a longitudinal study that there was discordance between areas of regional atrophy and areas of decreased blood flow. The explanations offered are that observed decline in blood flow in neocortex is in part explained by remote lesions (e.g. in entorhinal cortex), and secondly that in regions of primary damage, such as entorhinal cortex, loss of axons induces sprouting of the remaining nerve fibres replacing lost connections and maintaining synaptic activity, and hence blood flow.

A further clinical study (i.e. in phase 3) will permit a more detailed comparison between patterns of change produced by rember™ treatment which are visualised respectively by SPECT and PET. It is likely that PET reports changes which are closer to primary pathology, whereas SPECT reports remoter functional changes in regions that are functionally dependent on regions of primary pathology. The picture is further complicated by the fact that as the disease progresses, the remote functionally dependent cortical regions themselves become regions of advancing primary pathology. Therefore, the patterns of change over time and of response to Tau aggregation inhibitor treatment are likely to be complex.

Regardless of these potential complexities, the present discovery that MTL structures are metabolically highly responsive to Tau aggregation inhibitor therapy is an important finding. As indicated above, this provides strong evidence in support of the primary mechanism of action of rember™ therapy as specifically a Tau aggregation inhibitor treatment. An important practical implication of this finding is in the design of phase 3 trails to confirm rember™ as a disease modifying therapy of AD. The type of evidence provided here is exactly what is required of a surrogate marker of Tau aggregation inhibitor efficacy, and it is believed that this should be acceptable to regulatory authorities as an objective biological marker of disease-modifying efficacy.

A second important practical implication is that preliminary data supporting disease modifying efficacy can now be provided within a much shorter time-frame than required for studies using cognitive end-points such as change in ADAS-cog. As indicated in the present report, the PET changes in MTL metabolism could be demonstrated after only 18 weeks of treatment, and it is conceivable that this could be shortened to 12 weeks. In preclinical studies, it was shown that changes in neuropathology in transgenic mice were demonstrable after only 3 weeks of treatment. It is therefore possible to design a phase 3 plan that incorporates an early functional brain scan read-out.

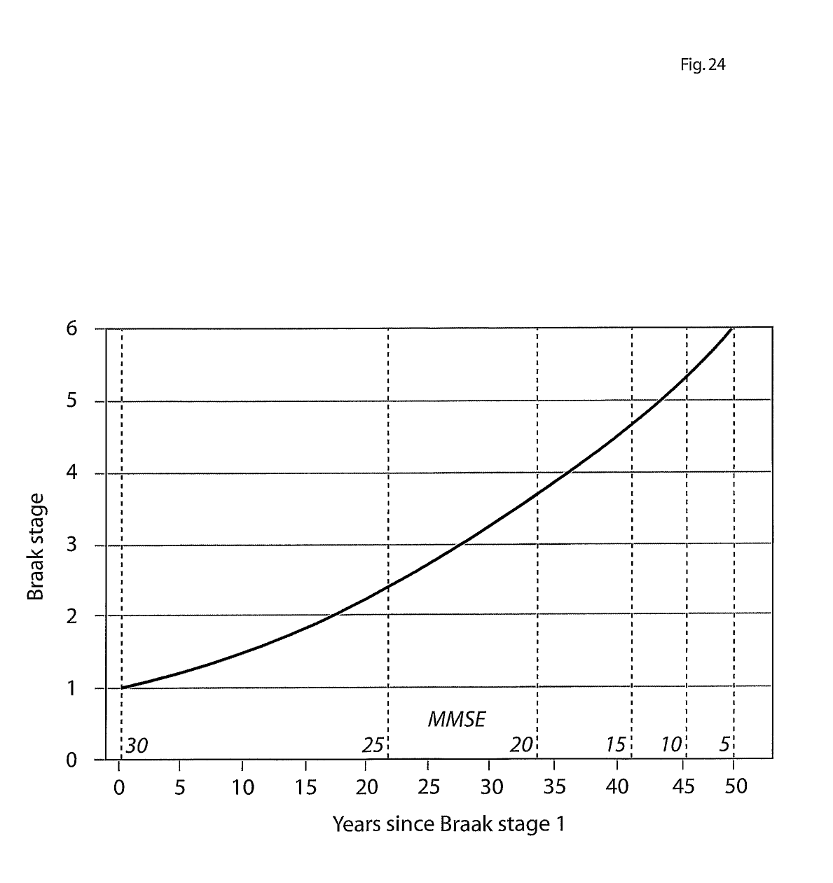

The third important practical implication of these findings is that they strongly support the potential for use of rember™ as an early stage preventive treatment of AD. As discussed above, irreversible damage occurs early in MTL structures. At Braak stages 2 and 3, there is already measurable tangle-mediated neuronal destruction in the MTL brain structures which are critical for memory and for higher order integrative functions via functional projections to temporo-parietal and orbito-frontal regions of the neocortex. As shown in FIG. 24, Braak stages 2 and 3 correspond approximately to the MMSE range 23-27, and overlap with early stages of cognitive impairment captured by the concept of Mild Cognitive Impairment (MCI).

The present evidence that it is possible to demonstrate selective metabolic enhancement within the MTL structures raises the possibility of undertaking a trial to prove efficacy in MCI in which the use of PET as a surrogate end-point plays a major role. There have been several high profile failures of clinical trials aiming to demonstrate efficacy of AD treatments in MCI. These have been very large, long-lasting and expensive studies, that have been bedevilled by non-random drop-out effects. The main reason is that conversion to AD has been the primary outcome measure. However, as the progression of the underlying pathology of AD is a very gradual process, it is very difficult to define categorical transitions accurately. Furthermore, it has been difficult to devise reliable cognitive tests which are sensitive to treatment effects. The present results offer for the first time the feasibility of using PET as a mechanistically sound surrogate outcome measure for early stage preventative intervention.

Conclusions from Functional Brain Scan Study

The functional brain scan study was a separate study nested within the rember™ clinical study. Functional brain scans performed two functions with the study: initial diagnostic classification, and as an independent physiological outcome measure. Two scanning modalities were used: SPECT (measuring regional cerebral perfusion) and PET (measuring glucose uptake). Both have been shown to be tightly linked to neuronal function, and both give comparable diagnostic findings.

The fundamental hypothesis underlying the scan study was based on the expectation that the neuropathological basis of the deficits seen in functional brain scans is specifically Tau aggregation pathology. The typical distribution of Tau aggregation pathology is very characteristic and highly stereotyped (Braak and Braak, 1991; Gertz et al., 1998) at both early pre-tangle and later stages (Lai, R. Y. K., Gertz, H.-J., Wischik, D. J., Xuereb, J. H., Mukaetova-Ladinska, E. B., Harrington, C. R., Edwards, P. C., Mena, R., Paykel, E. S., Brayne, C., Huppert, F. A., Roth, M. & Wischik, C. M. (1995) Examination of phosphorylated tau protein as a PHF-precursor at early stage Alzheimer's disease. Neurobiology of Aging, 16:433-445; Mukaetova-Ladinska, E. B., Garcia-Siera, F., Hurt, J., Gertz, H. J., Xuereb, J. H., Hills, R., Brayne, C., Huppert, F. A., Paykel, E. S., McGee, M., Jakes, R., Honer, W. G., Harrington, C. R. & Wischik, C. M. (2000) Staging of cytoskeletal and b-amyloid changes in human isocortex reveals biphasic synaptic protein response during progression of Alzheimer's disease. American Journal of Pathology, 157:623-636). As there is almost no tangle-mediated neuronal destruction in the neocortex until very late stage disease (Garcia-Sierra et al., 2001), it was surmised that the functional deficits detected by functional brain scan modalities such as SPECT and PET are largely due to functional impairment in neurons caused by pre-tangle oligomeric Tau aggregates. The critical prediction, therefore, was that a treatment such as rember™, which enhances clearance of Tau aggregates ought to prevent the functional decline measured by SPECT and PET scanning modalities. This prediction was borne out by the present study.

There are several important results to note in this study. The first is the well established correlation between baseline SPECT defect and baseline ADAS-cog score (Nebu et al., 2001). This was confirmed by the present study, and shown to be dominated by left temporo-parietal brain regions, as well as a small region in the frontal association cortex. Therefore, these are the brain regions which determine cognitive function as measured by ADAS-cog score. This scale is therefore particularly well suited to detection of dysfunction in the temporo-parietal regions affected in AD, which may explain why the ADAS-cog scale has become the de-facto gold standard in clinical trials in AD, despite the fact that it is not a suitable scale for use in routine clinical practice.

The most important conclusion from the study is that the normal decline trajectory seen as the progressive loss of brain function in temporo-parietal and frontal brain regions was entirely arrested by treatment with rember™. It appears extraordinary that the functional consequences of progressive accumulation of Tau aggregates can be measured over as short time-interval as 6 months. This underlines the fact that AD is truly a rapidly progressive disease in terms of loss of brain function, even though the underlying aggressiveness of the disease process does not appear on commonly used clinical metrics such as the MMSE scale. Slow rate of deterioration on the MMSE scale (1-2 units per annum) gives clinicians the mistaken impression that the disease does not progress rapidly. However, what appears on the psychometric scale is not a measure of the disease itself and is confounded by the extensive cognitive reserve capacity that is present in the brain which mitigates the visible clinical effects of advancing pathology.

This was borne out by the dissociation between the objective physical evidence 8% reduction in neuronal function, measured using blood flow, in CDR-mild subjects over the first 6 months of the study, and failure of this to register on a sensitive psychometric instrument such as ADAS-cog over the same period. However, decline seen on the functional brain scans was predictive of future clinical decline that emerged six months later in the CDR-mild group. This strongly supports the conclusion that psychometric measures of disease progression are confounded by cognitive reserve capacity, particularly at the stage of the disease captured within the CDR-mild category.

The converse of this is that the benefit of rember™ can be demonstrated much more sensitively by functional brain scan than by psychometric testing. Despite the fact that the benefit of rember™ treatment could not be demonstrated on any psychometric scale in CDR-mild subjects over the first 6-month period of the trial, the benefit was clearly demonstrated by physical measurement of brain function. Indeed, in CDR-mild subjects, there was evidence of improvement in function at the 30/60 mg doses of rember™.

This was further demonstrated by the data for the low (100 mg) dose. Although smaller than the functional brain scan benefit seen for the 30/60 mg doses, the low (100 mg) dose nevertheless had a significant benefit relative to placebo after 4 months of treatment. However, the low (100 mg) dose had no apparent beneficial impact on outcomes measured by any of the psychometric scales. Again, when measured over the longer time-course, the benefit of the low (100 mg) dose became apparent only over 50 weeks (data provided in Case 9). Therefore, just as decline on SPECT scan was predictive of future psychometric decline, so benefit on SPECT scan was predictive of future psychometric benefit.

Despite the dissociations between scan data and ADAS-cog data when measured within specific treatment and severity groups, there was overall a robust correlation between improvement in functional brain scan and improvement on ADAS-cog, reaching a correlation coefficient values in the range of −0.44 to −0.46, which is high in the field of clinical-imaging correlation studies. Indeed the correlations between the change scores were higher than the baseline correlation between ADAS-cog and scan deficit (r=0.21).

When comparing drug effects seen via psychometric scales and those seen by physical measurement of brain function, it should be borne in mind that scales such as ADAS-cog are themselves only proxy or surrogate outcome measures of treatment benefit in dementia. Dementia is a global brain disorder which results in severe impairment of the global functioning of the whole person. It is only in part a cognitive disorder. The introduction of global clinical outcome scales for regulatory purposes, such as the CGIC scale (e.g. European Agency for the Evaluation of Medicinal Products (1997) Note for Guidance on Medicinal Products in the Treatment of Alzheimer's Disease), represents an attempt to go beyond cognitive scales to capture this global dimension. It is likewise arguable that a physical measure of global brain function, such as SPECT scan, is also a superior and more objective measure of underlying disease process than cognitive scales.

The present results provide strong confirmation of the efficacy of rember™ at all active treatment doses in subjects who were CDR-mild at baseline over the first 6 months of treatment despite the failure to demonstrate efficacy using standard psychometric end-point measures in the same group. The failure to detect either placebo decline or treatment benefit over this period in CDR-mild subjects by any of the clinical psychometric measures used indicates the severe limitations of psychometric outcome measures at earlier disease stages in the face of cognitive reserve, and the powerful influence of cognitive reserve masking objectively demonstrable decline in brain function.

It is therefore concluded that disease-modifying efficacy of treatments, of which rember™ is the first available example, can be demonstrated even in the absence of cognitive decline in placebo-treated subjects as measured by conventional psychometric instruments. The method consists in measurement of decline in functional brain scan in subjects treated with placebo, using methods such as SPECT or PET, and comparing this subjects receiving active treatment. The benefit of treatment can be demonstrated by either ROI or SPM methods. The advantage of this method is that efficacy can be demonstrated over a relatively short time period, such as for example 4 months of treatment. In mild or early stage AD, demonstration of efficacy would require subjects to be kept on placebo for much longer periods, eg typically 1 year. However, as described in the next section, there are severe limitations in the conduct of longer-term studies in AD. It will be apparent to the skilled worker that other methods of objective determination of ongoing disease progression, such as those indicated in WO02/075318 could also be used. For the present, however, such methods are not yet routinely clinically available, whereas the methods described in the present specification are already available in routine clinical practice. The fact that such methods, hitherto generally used for diagnostic purposes, have applicability in the detection of therapeutic efficacy even in circumstances where there is no apparent clinical benefit of a disease-modifying treatment, is highly unexpected.

### Example 5

### Fit-Survivor Bias Limits the Conduct of Clinical Trials in AD Over Longer Intervals

A major difficulty in the conduct of trials aiming to demonstrate modification of disease progression over longer time courses is fit-survivor bias. Within a randomised design context, longer trials in which patients/carers perceive continuing deterioration engenders the problem of non-random drop-out over time. Non-random drop-out can inflate the apparent effect size of relatively ineffective drugs with prominent side effects, such as the AChE inhibitors, particularly in ITT/LOCF analyses where the last available observation is used to impute missing data (Gray, R., Stowe, R. L., Hills, R. K., Bentham, P. (2001) Non-random drop-out bias: intention to treat or intention to cheat? Controlled Clinical Trials, 22:38S-39S; Hills R, Gray R, Stowe R, Bentham P. (2002) Drop-out bias undermines findings of improved functionality with cholinesterase inhibitors. Neurobiology of Aging, 23:89; Lavori P W (1992) Clinical trials in psychiatry: should protocol deviation censor patient data? Neuropsychopharmacology, 6, 39-48; Little, R., Yau, L. (1996). Intent to treat analysis for longitudinal studies with drop outs. Biometrics, 52:1324-1333).

Because of progressive drop-out of subjects who decline over time (so-called “non-responders”), the surviving treated population will appear to have a lower rate of decline overall.

Fit survivor bias arises when the following conditions are met: (i) subjects feel they have to make a decision about continuing medication (in the rember™ study subjects were explicitly given the option of discontinuing after 24 weeks of treatment); (ii) the disease is progressive and subjects experience continuing decline; (iii) the medication has side effects. The standard approach to this problem is to undertake an ITT/LOCF analysis, in which the last available observation is used to impute a score at the final analysis time-point, on the assumption that initial randomisation is sufficient to deal with this source of bias.

Whereas the effect of fit-survivor bias using LOCF data imputation is thought to inflate apparent effect size for drugs such as the AChE inhibitors, the effect for a drug such as rember™, which stabilises disease progression, was to compress apparent effect size, particularly at the 50-week time point. This is because non-random drop-out occurs early in the active arms for the AChE inhibitors, whereas it occurred late in the placebo arm of the rember™ trial. Thus subjects in the Least Exposed Dose arm (ie subjects switched to the low (100 mg) dose after 6 months) who continued to decline withdrew from the study largely after 24 weeks. It is conceivable that this effect may have been compounded by the fact that the dose to which placebo subjects were switched during the second 6-months of the study (i.e., low (100 mg) bd) also produced adverse effects, most notably diarrhea, which was the main cause of withdrawal from the trial. However, biased withdrawal was not a factor in CDR-mild subjects in the same low (100 mg) comparator treatment arm. Therefore the primary driver of non-random withdrawal in the rember™ trial was the experience of continuing rapid decline which was most apparent in the CDR-moderate group.

Impact of Fit-Survivor Bias

A linear mixed effects analysis was undertaken to determine how time of discontinuation influenced the apparent rate of disease progression as measured by the ADAS-cog change score in all groups. The critical index variable is the significance of the interaction between observed change score at 50 weeks and time of discontinuation at or after 24 weeks. The significance values for the interaction terms are shown in Table 13.

TABLE 13
Significance (p-value) of interaction-term describing effect of early
withdrawal on change in ADAS-cog score relative to baseline

| Category | CDR-mild | CDR-moderate |
|---|---|---|
| placebo-low | 0.291 | 0.0025 |
| low (100 mg) | 0.644 | 0.0077 |
| 30 mg | 0.625 | 0.0611 |
| 60 mg | 0.738 | 0.148 |

As can be seen from Table 13, time of discontinuation did not affect rate of disease progression in patients who were CDR-mild at baseline, but was highly significant in patients who were CDR-moderate at baseline and who were in either the placebo-low or low (100 mg) arms. The effect had borderline significance in the 30 mg arm in moderate subjects. Thus, patients who feel they have done well in the trial are more likely to stay in the trial, whereas patients who have deteriorated are more likely to discontinue medication. Therefore, it is appropriate to apply a method of correction for fit-survivor bias. This is discussed further below.

Failure of LOCF Methodology to Correct for Fit-Survivor Bias

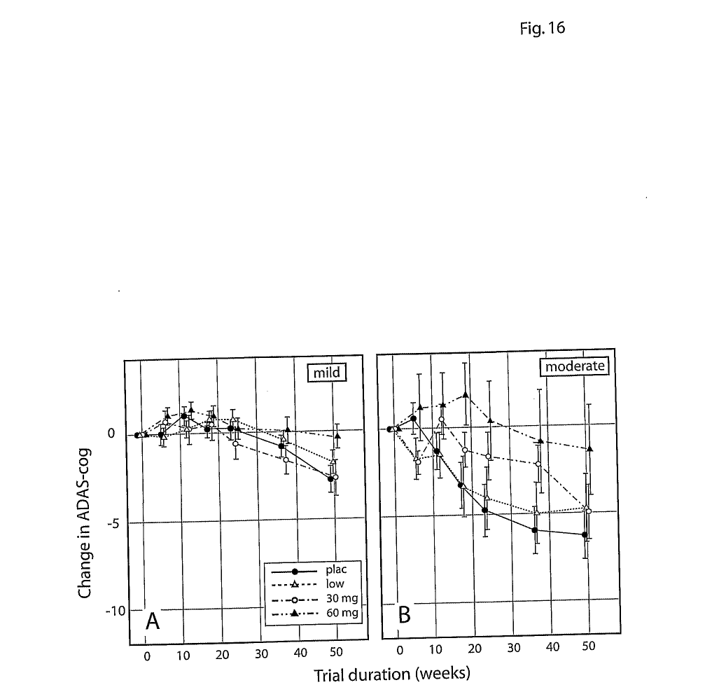

A mixed-effects analysis was conducted on subjects treated with rember™ over 50 weeks, and separating subjects by CDR severity at baseline. FIG. 16 shows the effect of the LOCF data imputation procedure. CDR-moderate subjects who were in the placebo-low and low (100 mg) arms show flattening after 24 weeks. Taken at their face value, these results would appear to suggest that subjects who are CDR-moderate at baseline cease to decline after 24 weeks, and that the disease stabilises, but only if they are not treated with a dose of rember™ that appears to be inactive in other analyses, notably analysis of treatment effect over the first 24 weeks. This appears to be highly implausible, and is also contrary to the published literature in that there are no reports to suggest that subjects who have more advanced disease cease to decline as a group provided they receive no or minimally active treatment. It is therefore concluded that the apparent flattening is an artifact of non-random drop-out of subjects who decline, i.e., fit-survivor bias.

Fit-Survivor Bias in Withdrawal from Active Treatment

A different line of evidence supporting the impact of fit-survivor bias was provided by data obtained from subjects who had withdrawn from prior treatment with AD-labelled drugs (principally AChE inhibitors) and who were then randomised to the placebo treatment arm in the rember™ study.

Psychometric Difference in Subjects Withdrawing from Active Treatment vs. Treatment-Naïve Subjects

The protocol of the rember™ study specified that subjects were not to be taking any concomitant AD-labelled medication (AChE inhibitors or memantine). Therefore, two types of subjects were randomised: those never previously treated with AD-labelled drugs (generally newly diagnosed), and those withdrawn for at least 6 weeks prior to randomisation from prior AD-labelled treatment (because of intolerance or lack of response). The distribution of subjects according to prior treatment status is shown in Table 14. As can be seen, 68% of subjects recruited to the rember™ trial were treatment-naïve.

TABLE 14
Prior treatment status of treatment group

| | Treated arm | | | | |
|---|---|---|---|---|---|
| | placebo- | | | | |
| Status | low | low(100 mg) | 30 mg | 60 mg | Total |
| Treated | 27 | 28 | 20 | 26 | 101 |
| Untreated | 65 | 62 | 39 | 55 | 221 |
| Total | 92 | 90 | 59 | 81 | 322 |

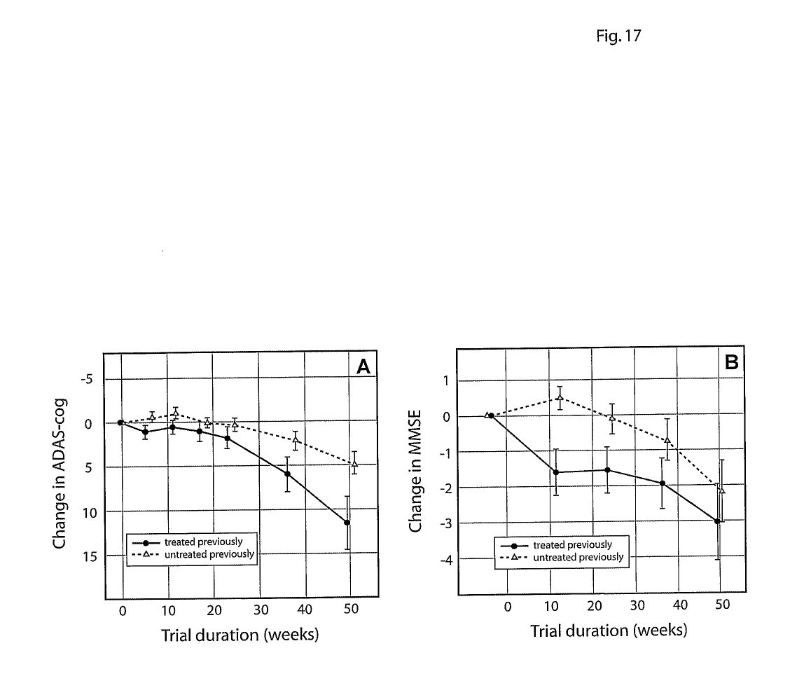

The rate of decline on the ADAS-cog and MMSE scales in subjects in the placebo-low arm was stratified according to prior treatment. This is shown in FIG. 17 and Table 15.

TABLE 15
Placebo-low decline by prior AD-labelled treatment

| | Estimate | 95% CI | p-value |
|---|---|---|---|
| ADAS-cog units (50 | | | |
| weeks) | | | |
| previously | 4.78 | 3.03, 6.53 | <0.0001 |
| untreated⁽¹⁾ | | | |
| previously treated⁽²⁾ | 10.53 | 8.05, 13.00 | <0.0001 |
| MMSE units (12 | | | |
| weeks) | | | |
| previously | 0.55 | −0.36, 1.47 | 0.232 |
| untreated⁽¹⁾ | | | |
| previously treated⁽²⁾ | −1.59 | −2.95, −0.23 | 0.0234 |

As shown in Table 15, the estimated overall 50-week decline in the placebo-low arm in subjects previously untreated with AD-labelled drugs was +4.8 ADAS-cog units. Subjects who had been treated previously declined by twice as much (10.5 units) over 50 weeks. A similar effect was found for the MMSE scores, although in this case the difference was significant only at 12 weeks (0.6 vs. −1.6 MMSE units; p-value, 0.0234).

Functional Brain Scan Difference in Subjects Withdrawing from Active Treatment vs. Treatment-Naïve Subjects

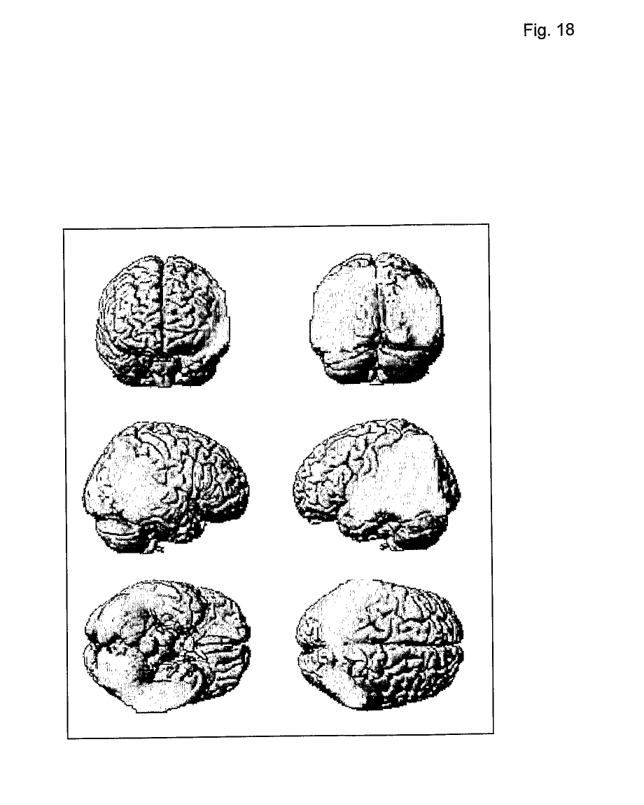

The same phenomenon was demonstrated by functional brain scan in the rember™ study. Subjects who entered the rember™ study after discontinuing prior treatment with AD-labelled drugs were found to have greater rCBF deficits in the temporal and parietal lobes than treatment-naïve subjects. The regions of significant difference are shown in FIG. 18. They show greater posterior spread of deficits, which is characteristic of more advanced AD.

Both the psychometric and the functional brain scan data indicate that subjects with a prior history of treatment with AD-labelled drugs showed more rapid decline when randomised to the placebo arm of the rember™ trial. This was also consistent with the evidence found in the primary ITT/OC analysis of the 24-week data that prior treatment with AD-labelled drugs was a significant cofactor indicating lower overall cognitive performance (see above).

It is concluded that the availability of approved AD-labelled drugs has a significant impact on patient selection for clinical trials of new drugs. Previously treated subjects are likely to be at a more advanced stage of the disease because of likely greater time since diagnosis. They are also more likely to have withdrawn from prior treatment as “unfit non-survivors”, i.e. subjects who continued to experience a combination of continuing decline and side-effects while taking AD-labelled drugs. This confirms the non-validity of long-term open-label studies purporting to establish the disease-stabilising effect of the currently available symptomatic treatments (Rogers, S. L., Doody, R. S., Pratt, R. D., Ieni, J. R. (2000). Long-term efficacy and safety of donepezil in the treatment of Alzheimer's disease: final analysis of a US multicentre open-label study. European Neuropsychopharmacology, 10:195-203.; Wallin, A. K., Andreasen, N., Eriksson, S., Batsman, S., Nasman, B., Ekdahl, A., Kilander, L., Grut, M., Ryden, M., Wallin, A., Jonsson, M., Olofsson, H., Londos, E., Wattmo, C., Eriksdotter Jonhagen, M., Minthon, L., Swedish Alzheimer Treatment Study Group (2007). Donepezil in Alzheimer's disease: what to expect after 3 years of treatment in a routine clinical setting. Dementia and Geriatric Cognitive Disorders, 23:150-160.). Subjects who remain on treatment represent a non-random selection of patients biased in favour of subjects who inherently have a lower rate of decline irrespective of treatment with AChE inhibitors.

### Example 6

### Linear Imputation Approach as a Method to Provide Unbiased Correction in the Conduct of Longer-Term (50-Week) Studies Aiming to Demonstrate Efficacy of Drugs as Disease-Modifying Agents

A method has been developed by the inventors which has not been described previously which permits unbiased correction for the fit-survivor phenomenon. Having demonstrated that it is appropriate and necessary to apply an imputation method to correct for fit-survivor bias, the following method was adopted to permit inbiased analysis of change in cognitive function over 50 weeks. If a patient who was CDR-moderate at baseline discontinued medication at some time after 24 weeks, a straight line is fitted to the graph of available ADAS-cog change scores against visit-date for the available data for each subject. The line was not forced to pass through zero, to allow for placebo effect, and because the first visit is subject to random fluctuations. The use of a straight-line fit per-subject is supported by the large studies of Stern et al., (1994), Doraiswamy et al., (2001) and Winblad, B., Wimo, A., Engedal, K., Soininen, H., Verhey, F., Waldemar, G., Wetterholm, A. L., Haglund, A., Zhang, R., Schindler, R. (2006) 3-year study of donepezil therapy in Alzheimer's disease: effects of early and continuous therapy. Dementia and Geriatric Cognitive Disorders, 21:353-363), which suggest that the general rate of decline is approximately linear in the score range 15 to 45 ADAS-cog units. The same approach can be applied to other outcome measures for which there were assessments scheduled after 24 weeks.

Analysis of Disease-Modifying Treatment Efficacy Over 50 Weeks

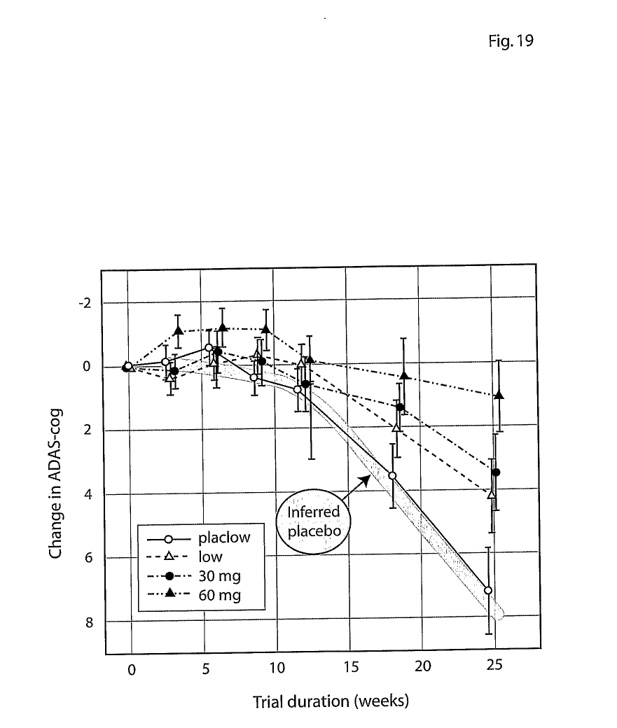

The 50-week study extended and confirmed the findings of the 24-week study study discussed above, and demonstrated significant benefits in both CDR-mild and CDR-moderate subjects in the overall ITT/OC and ITT/LOCF populations (FIG. 19; Tables 18 & 19). Subjects originally randomized to placebo were switched to the low (100 mg) dose bd after 24 weeks. This is referred to as the “placebo-low” treatment arm. Because of the minimal efficacy of the low (100 mg) dose on any of the psychometric scales over the first 24 weeks of treatment, the placebo-low treatment arm conveniently served as the Least Exposed Dose comparator arm for the 50-week study.

FIG. 19 shows the change from baseline in ADAS-cog score over 50 weeks. In this chart, the labelling conventions of “placlow” refers to subjects randomised to placebo and changed to low (100 mg) bd after 24 weeks, “low” refers to low (100 mg) dose tid, “30 mg” refers to 30 mg dose tid and “60 mg” refers to 60 mg dose tid. The shaded lines are best-fits calculated using the linear mixed effects random coefficients model and the bold grey line represents the inferred placebo unless stated otherwise. As can be seen, the response to rember™ occurs in two phases. For the 60 mg tid dose, there is initial improvement in the first 18 weeks, and after 24 weeks there is stabilisation of disease progression. For the other doses, there is also a difference between 0-24 weeks and 24-50 weeks response.

The mean decline observed over the 50-week study in placebo-treated subjects was 7.8 ADAS-cog units (FIG. 19). For subjects treated with rember™ at a dose of 60 mg tid, the decline seen over 50 weeks was not significantly different from zero on either the ADAS-cog scale or the MMSE scale for subjects (data not shown). On the ADAS-cog scale, about 60% of subjects improved or stayed the same at 50 weeks. On the MMSE scale, 62% improved or stayed the same at 50 weeks. The odds of a patient not declining on either scale were about 3.4 times better at the 60 mg dose than on placebo-low. The corresponding effect sizes were −6.8 ADAS-cog units and 3.2 MMSE units over the 50-week trial. In addition to the effect on disease progression, there was an initial symptomatic improvement at 15 weeks of 1.6 ADAS-cog units and 0.8 MMSE units at the 60 mg dose, comparable to that observed with AChE inhibitors.

TABLE 18
Effect sizes inferred from mixed effects analysis at 50 weeks (in
ADAS-cog units)

| Dose | Estimate | 95% CI | p-value⁽¹⁾ |
|---|---|---|---|
| low (100 mg) | −4.04 | −7.21, −0.87 | 0.0124 |
| 30 mg | −3.87 | −6.90, −0.84 | 0.0126 |
| 60 mg | −6.78 | −9.74, −3.82 | <0.0001 |

⁽¹⁾The p-value is from a test of whether the value is significantly different from placebo.

TABLE 19
Effect sizes inferred from least-squares analysis at 50 weeks (in
ADAS-cog units)

| Dose | Estimate | 95% CI | p-value⁽¹⁾ |
|---|---|---|---|
| low (100 mg) | −3.59 | −5.81, −1.37 | 0.0015 |
| 30 mg | −4.37 | −6.83, −1.92 | 0.0005 |
| 60 mg | −6.50 | −8.89, −4.14 | <0.0001 |

⁽¹⁾The p-value is from a test of whether the value is significantly different from placebo.

There was no deterioration on the non-cognitive scales in CDR-mild subjects in the placebo-low arm over 50 weeks. The non-cognitive outcomes at 50 weeks in CDR-moderate subjects confirmed the findings of the 24-week analyses. The NPI (Neuropsychiatric Inventory) demonstrated benefits for rember™ treatment over 50 weeks. Whereas subjects in the placebo-low arm declined by 9.6 units on the patient-disturbance scale and 4.9 units on the carer-distress scale, no such decline was seen in subjects continuously treated with rember™ over 50 weeks, with corresponding best effect sizes of −9.2 units and −4.6 units.

The placebo-low arm compared to the low (100 mg) arm provided a close approximation to a delayed start design to confirm that rember™ is disease modifying in a formal regulatory sense. Subjects who began later on a dose of minimal apparent therapeutic efficacy as judged by ADAS-cog over the initial 24 weeks remained significantly different at 50 weeks relative to subjects who had been receiving the low (100 mg) dose continuously. Furthermore subjects treated continuously at the low (100 mg) dose showed retardation in the rate of disease progression. This is shown in Tables 20 & 21. As expected, the effect sizes are somewhat smaller than those obtained when an inferred placebo method is used (as in Tables 18 & 19) to correct for the small effect of delayed-start treatment with the low (100 mg) dose in the second half of the study period.

TABLE 20
Estimated change from baseline Least Exposed Dose
comparator method

| (In ADAS-cog units) | Estimate | 95% CI | p-value⁽¹⁾ |
|---|---|---|---|
| placebo-low | 6.75 | 5.47, 8.03 | <0.0001 |
| low (100 mg) | 4.00 | 2.61, 5.38 | <0.0001 |
| 30 mg | 3.95 | 2.30, 5.61 | <0.0001 |
| 60 mg | 1.04 | −0.48, 2.56 | 0.179 |

⁽¹⁾The p-value is from a test of whether the value is significantly different from zero.

TABLE 21
Estimated effect size, Least Exposed Dose comparator method

| (In ADAS-cog units) | Estimate | 95% CI | p-value⁽¹⁾ |
|---|---|---|---|
| low (100 mg) | −2.76 | −4.64, −0.87 | 0.0043 |
| 30 mg | −2.80 | −4.89, −0.71 | 0.0089 |
| 60 mg | −5.71 | −7.70, −3.72 | <0.0001 |

⁽¹⁾The p-value is from a test of whether the value is significantly different from placebo.

Although there was a difference in the capsule dosage regime between the two arms (tid vs. bd), haematological side effects, which showed a clear dose-response profile, were indistinguishable with regard to the two dosing regimes, supporting the approximate equivalence of biological exposure (as discussed in prior-filed, unpublished US provisional application of Wischik, dated 3 October, Attorney Docket: 088736-0116), and hence supporting the inference that rember™ is disease-modifying. This is also confirmed by rember™'s ability to arrest disease progression over 50 weeks at the 60 mg dose, and reduced the rate of disease progression at the 30 mg and low (100 mg) doses at 50 weeks.

Demonstration that Method of Correction for Fit-Survivor Bias Does Not Inflate Effect Size

The purpose of these analyses was to demonstrate that the method used to correct for fit-survivor bias does not inflate effect size. In order to do this, it is first necessary to circumvent the problem of non-decline of CDR-mild subjects in the placebo arm over the first 24 weeks of the study. This was done as follows. Since CDR-mild subjects originally randomised to the placebo arm were found to decline over weeks 24 to 50 (see FIG. 2) after being switched to the low (100 mg) bd dose, this provided a basis for examining rember™'s efficacy over a similar period to that observed during the first 24 weeks of the study, but in a context where there was evidence of decline in CDR-mild subjects. For this purpose, the low (100 mg) arm provided a suitable comparator group as the Least Exposed Dose of rember™ dose. In fact, the analysis over the first 24 weeks in CDR-moderate subjects showed that the low (100 mg) dose had no effect on ADAS-cog over the first 24-weeks treatment period, and that the decline seen in subjects treated at this dose over this interval did not differ from true placebos, confirming that the low (100 mg) dose could be used as a comparator dose arm over a 6-month treatment period.

For this purpose, an approach which we term “mild-brought-forward confirmatory analysis” has been used. In this analysis, observations in the CDR-mild group for weeks 24 to 50 were brought forward to weeks 0 to 24. The original data for CDR-moderates over weeks 0 to 24 was used. In this analysis, placebos in the moderate group were true placebos, whereas the comparator dose in the milds was a delayed start low (100 mg) bd dose. Both were treated as placebo for this analysis. Two analyses are presented: 1) an analysis of the entire population comprising original moderates and milds-brought-forward; 2) an analysis of the subjects split by CDR-severity at baseline. Both analyses used the mixed effects model.

i) Mild-Brought-Forward Confirmatory Analysis: Main Analysis

This analysis uses a mixed-effects model with a random per-patient coefficient and a fitted straight line response curve. Since this analysis combines 24-week data from moderates, and 26-week data from milds, the inferred estimates shown in Tables 22 & 23 are calculated at 25 weeks.

TABLE 22
Mild-brought-forward confirmatory analysis, overall change
from baseline over 25 weeks

| (in ADAS-cog units) | Estimate | 95% CI | p-value⁽¹⁾ |
|---|---|---|---|
| placebo/placebo- | 4.65 | 3.49, 5.81 | <0.0001 |
| low | | | |
| low (100 mg) | 3.81 | 2.58, 5.04 | <0.0001 |
| 30 mg | 2.26 | 0.80, 3.73 | 0.0026 |
| 60 mg | 0.78 | −0.58, 2.18 | 0.256 |

⁽¹⁾The p-value is from a test of whether the value is significantly different from zero.

TABLE 23
Mild-brought-forward confirmatory analysis, overall effect size
over 25 weeks

| (in ADAS-cog units) | Estimate | 95% CI | p-value⁽¹⁾ |
|---|---|---|---|
| low (100 mg) | −0.84 | −2.54, 0.86 | 0.330 |
| 30 mg | −2.39 | −4.25, −0.52 | 0.0126 |
| 60 mg | −3.85 | −5.66, −2.04 | <0.0001 |

⁽¹⁾The p-value is from a test of whether the value is significantly different from placebo/placebo-low.

ii) Mild-Brought-Forward Confirmatory Analysis: Change and Effect Size Over 25 Weeks in CDR-Mild and CDR-Moderate Subjects

In this analysis the treatment responses and effect sizes at 50 weeks are split according to CDR severity at baseline. The analysis uses a mixed-effects model with a random per-patient coefficient and a fitted straight line response curve. Table 24 shows change from week 24 to week 50, and Table 25 shows effect size at week 50.

TABLE 24
Mild-brought-forward change from baseline over 25 weeks in
mild and moderate subjects

| | CDR-mild | | | CDR-moderate | | |
|---|---|---|---|---|---|---|
| | change over | | | change over | | |
| (In ADAS- | 25 weeks | | | 25 weeks | | |
| cog units) | Estimate | 95% CI | p-value⁽¹⁾ | Estimate | 95% CI | p-value⁽¹⁾ |
| placebo/ | 4.26 | 2.91, 5.61 | <0.0001 | 5.37 | 3.09, 7.65 | <0.0001 |
| placebo-low | | | | | | |
| low (100 mg) | 3.60 | 2.30, 4.91 | <0.0001 | 4.82 | 1.60, 8.04 | 0.0040 |
| 30 mg | 3.07 | 1.26, 4.88 | 0.0010 | 1.04 | −1.46, 3.54 | 0.410 |
| 60 mg | 1.07 | −0.41, 2.56 | 0.156 | 0.30 | −3.62, 3.01 | 0.855 |

⁽¹⁾The p-value is from a test of whether the value is significantly different from zero.

TABLE 25
Mild-brought-forward effect size over 25 weeks in mild and moderate subjects

| (In ADAS- | CDR-mild effect size | | | CDR-moderate effect size | | |
|---|---|---|---|---|---|---|
| cog units) | Estimate | 95% CI | p-value⁽¹⁾ | Estimate | 95% CI | p-value⁽¹⁾ |
| low(100 mg) | −0.66 | −2.53, 1.22 | 0.490 | −0.54 | −4.49, 3.40 | 0.783 |
| 30 mg | −1.19 | −3.44, 1.06 | 0.299 | 4.33 | −7.71, 0.95 | 0.0131 |
| 60 mg | −3.19 | −5.19, −1.19 | 0.0020 | −5.67 | −9.69, −1.65 | 0.0065 |

⁽¹⁾The p-value is from a test of whether the value is significantly different from placebo/placebo-low.

iii) Conclusion

The mild-brought-forward confirmatory analyses do not rely on the linear data imputation used to correct to fit-survivor bias. Effect sizes calculated over 25 weeks in this manner for both ADAS-cog and MMSE (data not shown) were greater than half the effect sizes determined directly from the 50-week analyses. Since disease progression has been shown to be linear over 12 months (Stern et al., 1994), it would be expected that the placebo-decline and effect size of treatment that would be estimated to occur at 25 weeks from an analysis conducted at 50 weeks would be approximately half those observed at 50 weeks. However, direct analyses of 25 week placebo-decline and treatment effect using the mild-brought-forward approach showed placebo-decline and treatment effect which were more than half the corresponding results estimated at 50 weeks. But the mild-brought-forward analysis does not require on the linear-imputation method used to correct for fit-survivor bias, whereas the 50-week results do make use of linear-imputation. Therefore, the used of linear imputation to correct for fit-survivor bias does not inflate the estimated effect size. Indeed, if anything, it is conservative.

### Example 7

### Importance of Demonstrating Disease-Modifying Efficacy at Early Braak Stages of Neurodegeneration in AD

Braak Staging

Braak staging is a key organizing concept for relating the molecular foundations of Tau aggregation to disease progression observed clinically. In contrast to the widely used clinical scales which measure mainly syndromal AD, Braak staging is a pathological characterization based on the progression of Tau pathology. The defining Braak study (Braak, H. & Braak, E. (1991) Neuropathological staging of Alzheimer-related changes. Acta Neuropathologica 82:239-259) observed that the distribution pattern and the packing density of neurofibrillary tangles throughout the brains of both AD and non-demented patients had only minor inter-individual variations. Braak found that neurofibrillary tangles first formed in the entorhinal region of the brain followed by the limbic and neocortex regions and hence identified six distinct stages (“Braak stages”) of this progression. Clinical progression of AD is closely linked to Braak staging, which is itself a measure of anatomical progression, thereby lending further credence to the role of Tau aggregates in clinical dementia.

By contrast, the Braak study found that the distribution pattern and packing density of Aβ deposits had limited significance for the differentiation of neuropathological stages. Furthermore, accumulations of Aβ plaques were also frequently found in the cortex of non-demented individuals.

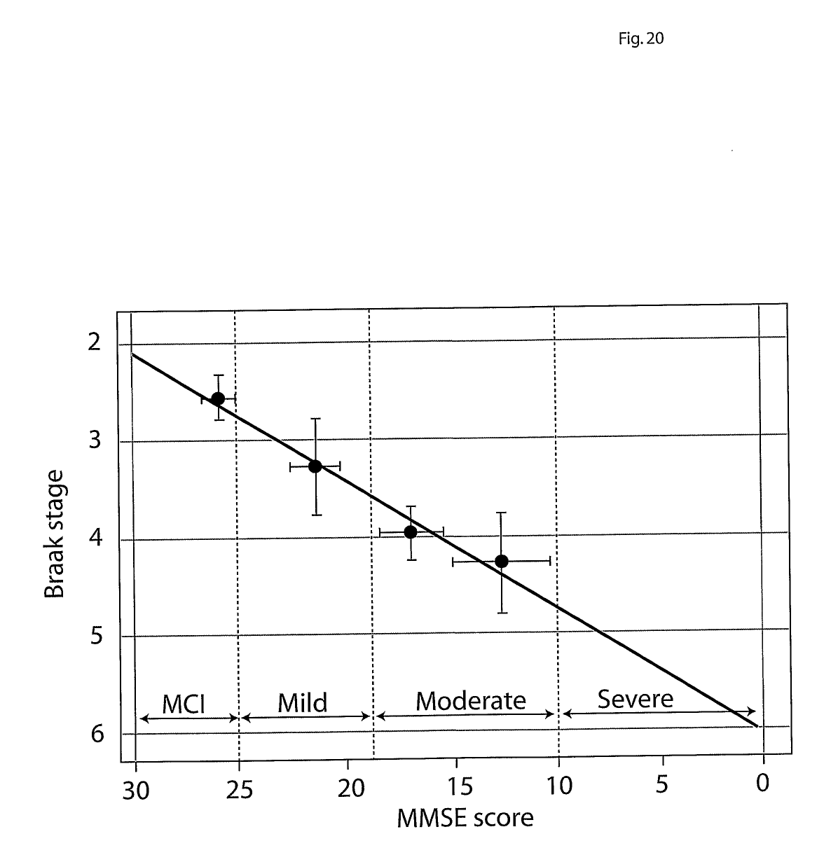

The relationship between Braak stages and cognitive decline was further confirmed in later studies (Gertz, H.-J., Xuereb, J., Huppert, F., Brayne, C., McGee, M. A., Paykel, E., Harrington, C., Mukaetova-Ladinska, E., Arendt, T. & Wischik, C. M. (1998) Examination of the validity of the hierarchical model of neuropathological staging in normal aging and Alzheimer's disease. Acta Neuropathologica 95:154-158; Mukaetova-Ladinska, 2000). In these studies, the workers examined neurofibrillary pathology in the brain, measured by Braak stage, dementia severity and the last recorded MMSE score, of 48 subjects who had been followed clinically while still alive. FIG. 20 sets out the approximate correlation between MMSE score and Braak stage.

Braak Staging and Aging

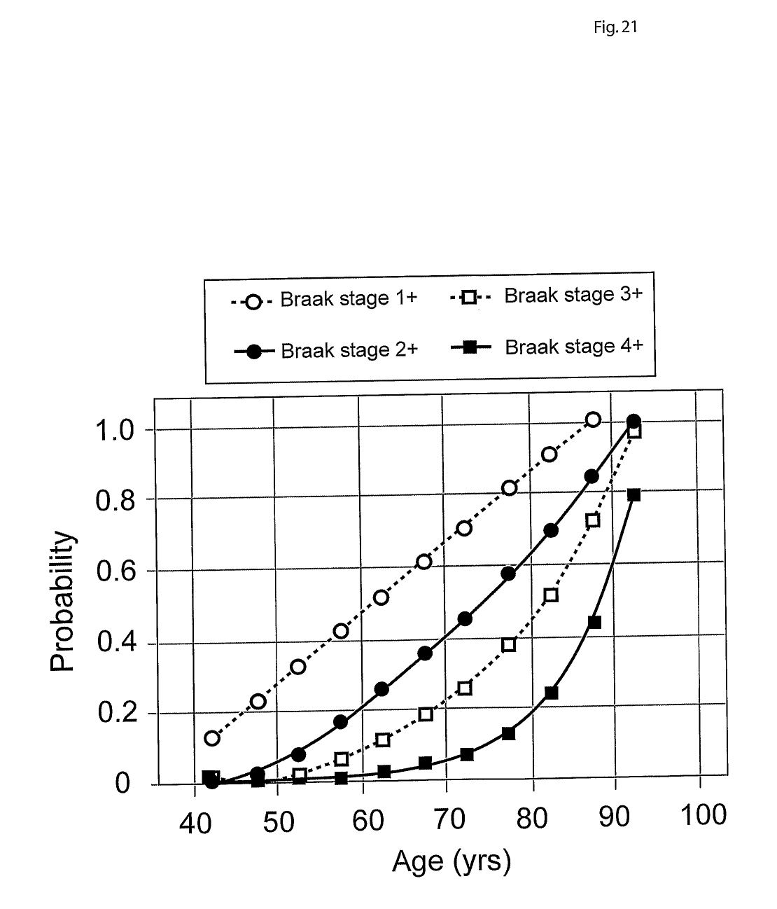

In a further study from the Braak group (Ohm, T. G., Müller, H., Braak, H. & Bohl, J. (1995) Close-meshed prevalence rates of different stages as a tool to uncover the rate of Alzheimer's disease-related neurofibrillary changes. Neuroscience 64:209-217), the brain tissues from 887 subjects from ages 40-90 were examined post-mortem. The inventors used this data to conduct a survival analysis permitted derivation of age-specific probabilities of Braak-stage transitions. As shown in FIG. 21 below, age is highly correlated with Braak staging. There is progressive migration towards higher Braak stages with increasing age.

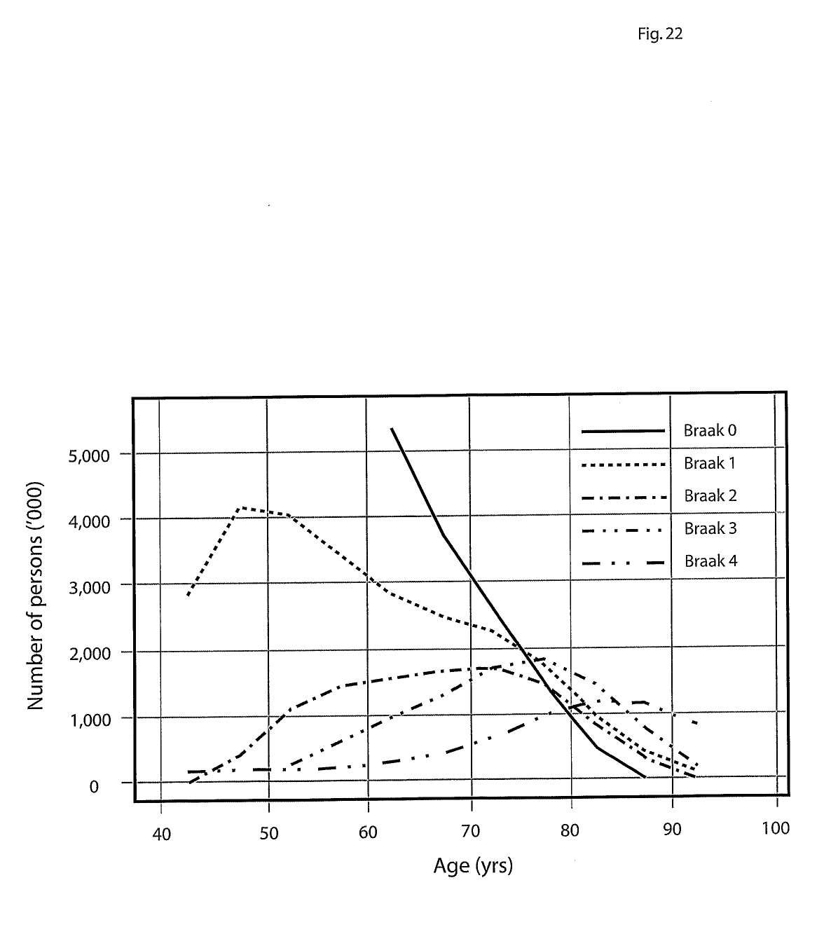

The age-specific probabilities of Braak stage transitions were applied to U.S. population data (United Nations World Population Prospects: The 2004 Revision, Volume III: Analytical Report; http://www.un.org/esa/population/publications/WPP2004/wpp2004.htm) to calculate the expected number of persons at each Braak stage by age. This is shown below in FIG. 22. As can be seen, there is a progressive transition with increasing age, from Braak stage 1, which peaks at about age 50, to Braak stage 4 or beyond, which peaks at about age 85, over a time-span of 30-40 years.

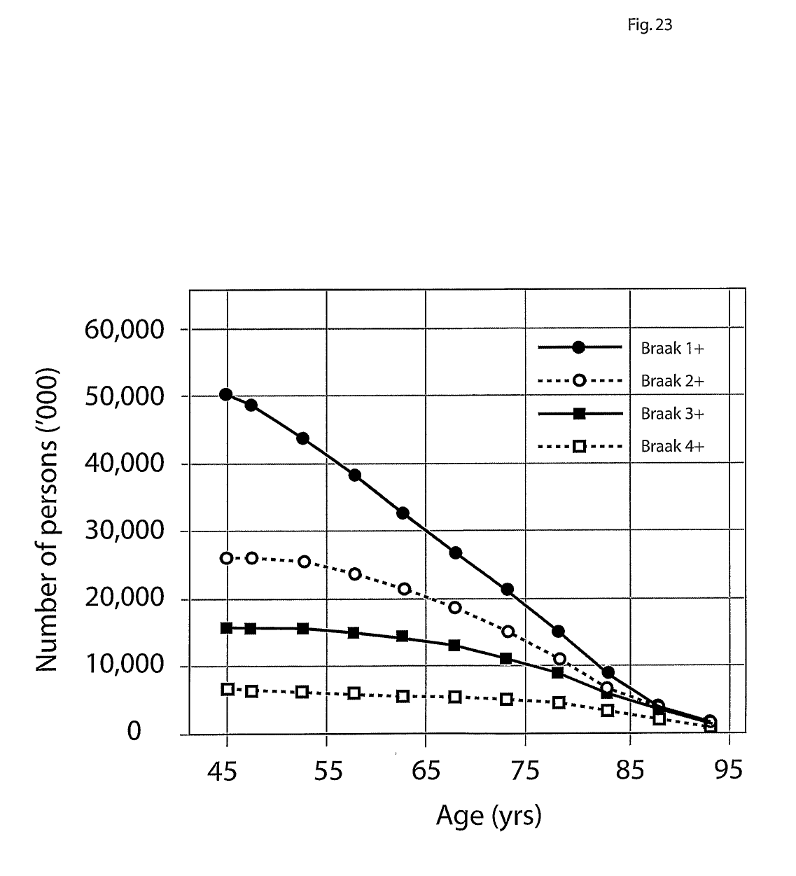

The same analysis was used to calculate the cumulative number of individuals in the U.S. at or above a given Braak stage threshold (FIG. 22). From the age-distribution shown in FIG. 23 below, there are over 25 million individuals over the age of 45 in the U.S. who are at Braak stage 2 or greater.

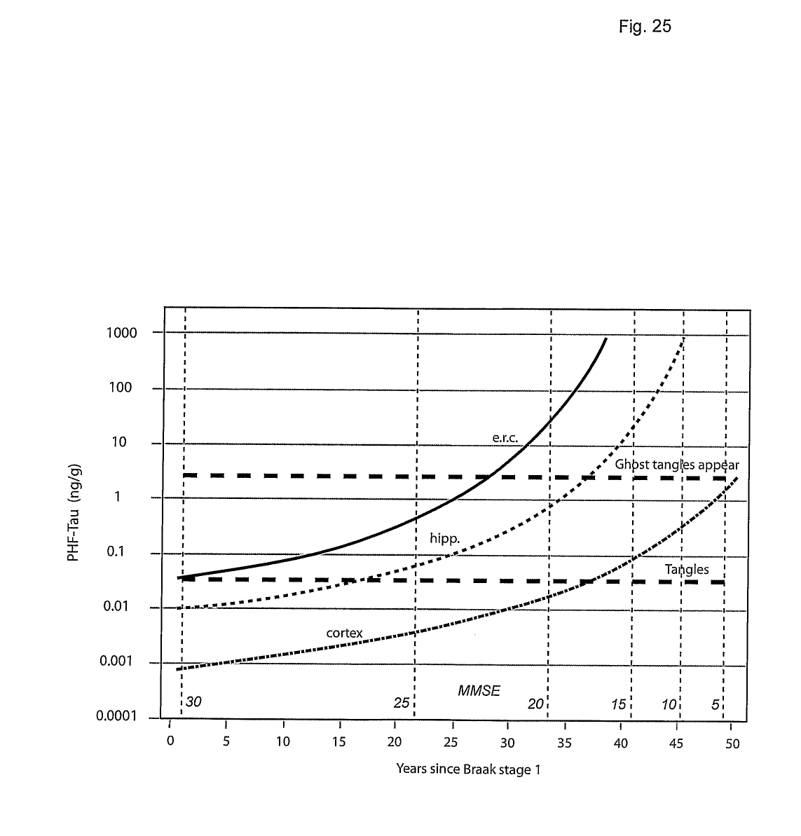

Further, using their own analysis of the Ohm et al. (1995) study, the Medical Research Council Study (The Medical Research Council Cognitive Function and Aging Study (MRC CFAS). (1998) Cognitive function and dementia in six areas of England and Wales: The distribution of MMSE and prevalence of GMS organicity level in the MRC CFA study. Psychological Medicine 28:319-335), data from Mukaetova-Ladinska, E. B., Garcia-Sierra, F., Hurt, J., Gertz, H. J., Xuereb, J. H., Hills, R., Brayne, C., Huppert, F. A., Paykel, E. S., McGee, M., Jakes, R., Honer, W. G., Harrington, C. R. & Wischik, C. M. (2000) Staging of cytoskeletal and b-amyloid changes in human isocortex reveals biphasic synaptic protein response during progression of Alzheimer's disease. American Journal of Pathology 157:623-636; Lai, R. Y. K., Gertz, H.-J., Wischik, D. J., Xuereb, J. H., Mukaetova-Ladinska, E. B., Harrington, C. R., Edwards, P. C., Mena, R., Paykel, E. S., Brayne, C., Huppert, F. A., Roth, M. & Wischik, C. M. (1995) Examination of phosphorylated tau protein as a PHF-precursor at early stage Alzheimer's disease. Neurobiology of Aging 16, 433-445; and Garcia-Sierra, F., Hauw, J. J., Duyckaerts, C., Wischik, C. M., Luna-Mu\check{n}oz, J. & Mena, R. (2000) The extent of neurofibrillary pathology in perforant pathway neurons is the key determinant of dementia in the very old. Acta Neuropathologica 100:29-35, the inventors produced an overall map of Tau aggregation, Braak staging and MMSE decline shown in FIGS. 24 and 25.

As can be seen from FIG. 24, the progression from Braak stage 1 to Braak stage 6 can be estimated to take approximately 50 years. Similarly, the transition MMSE scores from 30 (full-scale) to an MMSE score of less than 20 occurs at approximately Braak stage 4 and takes approximately 30 years after the transition to Braak Stage 1.

FIG. 25 shows a mapping of the measured brain level of aggregated Tau (in nanogram of aggregated Tau per gram of brain issue) in three representative brain regions: “erc”—entorhinal cortex; “hipp”—hippocampus; “cortex”—frontal and temporal neocortex. From the logarithmic scale in FIG. 25, it can be seen that the level of aggregated Tau in the brain increases exponentially over time. Furthermore, aggregated Tau accumulation in the brain has a non-linear relationship with MMSE score. A drop of 5 MMSE units from 30 to 25 corresponds to a 7-fold increase in PHF levels. A further 5 unit drop to 20 corresponds to a 16-fold increase. A further 5 unit drop to 15 corresponds to a 56-fold increase in PHF-levels. These levels continue to increase up to an upper threshold. This corresponds approximately to the level at which “ghost tangles” first appear in the corresponding brain region representing neuronal death via tangle formation. Tangle-mediated neuronal death is a relatively late stage of the process, which occurs well after the onset of functional impairment discussed previously. In the neocortex, for example, there is evidence of Tau aggregation and loss of neuronal function from Braak stage 2 onwards (see earlier section Tau aggregation and loss of synaptic function), whereas tangle-mediated cell death is not seen until Braak stage 6. These early functional changes are potentially reversible by Tau aggregation inhibitor therapy.

It has been shown that the entorhinal cortex and hippocampus are early casualties of the disease process, and that neuronal destruction occurs early in these regions (Gertz et al., 1998; García-Sierra, F., Wischik, C. M., Harrington, C. R., Luna-Mu\check{n}oz, J. & Mena, R. (2001) Accumulation of C-terminally truncated tau protein associated with vulnerability of the perforant pathway in early stages of neurofibrillary pathology in Alzheimer's disease. Journal of Chemical Neuroanatomy 22:65-77). These brain regions are essential for memory, and explain why memory impairment is such a prominent feature of early stage disease. Because of the early vulnerability of these regions, which may reach the stage of tangle-mediated cell death before any clinical symptoms are detected in individuals with high cognitive reserve (i.e., highly educated individuals), it is clearly important to initiate as early as possible treatment that could minimise irreversible damage.

As shown in FIG. 25, it can be seen that, in the general population, damage to entorhinal cortex becomes irreversible as early as MMSE score 23 units, when the appearance of ghost tangles indicates the onset of the stage of tangle-mediated neuronal death. Damage to hippocampus becomes irreversible at MMSE score 18 units. Accumulation of PHFs in the neocortex occurs much later and more slowly. In the this model, the extent of tangle-mediated neuronal destruction is low in neocortex even at the later stages of the disease. These regional differences highlight the danger of relying on the clinical instruments currently in widespread use to determine when to initiate treatment of the kind offered by rember™. As discussed in Example 4, such instruments give a misleading impression that the degree of clinical impairment is minor or even non-existent. This was strongly supported in the rember™ Phase 2 study, where it was shown that individuals classified at mild on the CDR scale showed no evidence on decline on any psychometric scale over 6 months, but nevertheless lost 8% of neuronal function shown by functional brain scan over the same time (see Example 4). In the entorhinal cortex and hippocampus, the damage underlying clinically minor deficits is in fact terminal.

The most important general conclusion from this Example is that there is a considerable period of time that can be detected clinically as mental impairment above an MMSE score of ˜25 where there is good prospect of treatment before irreversible damage occurs in the brain structures critical for memory function. These stages correspond clinically to so-called MCI and Mild AD. This provides a strong rationale for introduction of preventative treatment as early as possible in the disease. In particular, this provides a strong rationale for development of methods for proving the efficacy of drugs typified by rember™ even in circumstances such as early stages of AD and related disorders when conventional approaches may fail to provide evidence of treatment efficacy.

1. A method for assessing the efficacy of a pharmaceutical which is putatively disease modifying of a cognitive disorder, for use in the treatment or prophylaxis of that cognitive disorder, the method comprising the steps of:
(1) stratifying a subject group into at least 2 sub-groups according to a baseline indicator of likely disease progression,
(2) treating members of each subject group with the pharmaceutical for a treatment time frame,
(3) deriving psychometric and optionally physiological outcome measures for each treated patient group,
(4) comparing the outcomes at (3) with a comparator arm of said sub-groups which is optionally a placebo or minimal efficacy comparator arm,
(5) using the comparison in (4) to derive an efficacy measure for the pharmaceutical.

2. A system for assessing the efficacy of a pharmaceutical which is putatively disease modifying of a cognitive disorder, for use in the treatment or prophylaxis of that cognitive disorder, the system comprising the steps of:
(1) stratifying a subject group into at least 2 sub-groups according to a baseline indicator of likely disease progression,
(2) selecting a treatment time frame over which members of each subject group are to be treated with the pharmaceutical,
(3) selecting psychometric and optionally physiological outcome measures to be derived for each treated patient group and a comparator arm of said sub-groups which is optionally a placebo or minimal efficacy comparator arm,
whereby the efficacy measure for the pharmaceutical may be derived from a comparison of the treated patient groups and the comparator arm.

3. A method as claimed in claim 1 wherein the cognitive disorder is a neurodegenerative disorder causing dementia.

4. A method as claimed in claim 3 wherein the pharmaceutical is a putative inhibitor of pathological protein aggregation, where the aggregation is associated with the neurodegeneration.

5. A method as claimed in claim 3 wherein the neurodegenerative disorder is a tauopathy.

6. A method as claimed in claim 5 wherein the neurodegenerative disorder is selected from: Alzheimer's disease, MCI, motor neurone disease, Fronto-temporal dementia, Lewy body disease, Pick's disease, Progressive Supranuclear Palsy.

7. A method as claimed in claim 6 wherein the neurodegenerative disorder is Alzheimer's disease or MCI and the psychometric measures are selected from the group consisting of: Alzheimer's Disease Assessment Scale-cognitive subscale (ADAS-cog), National Institute of Neurological and Communicative Disorders and Stroke-Alzheimer's Disease and Related Disorders Association (NINCDS-ADRDA), Diagnostic and Statistical Manual of Mental Disorders, 4th Edn (DSMIV).

8. A method as claimed in claim 3 wherein the baseline indicator into which the sub-groups are stratified is disease severity using the Clinical Dementia Rating (CDR) scale.

9. A method as claimed in claim 8 wherein the sub-groups are subjects having a CDR rating of 1 (mild sub-group) or 2 (moderate sub-group).

10. A method as claimed in claim 3 wherein the sub-group having the lower disease severity is tested for a longer timeframe than the sub-group having the higher disease severity.

11. A method as claimed in claim 10 wherein the sub-group having the lower disease severity is tested for greater than 12 months.

12. A method as claimed in claim 3 wherein the sub-group having the lower disease severity is tested for a treatment time-frame over which there is no significant clinical decline.

13. A method as claimed in claim 12 wherein the sub-group having the lower disease severity is tested for less than 9, 6, 5, 4, or 3 months.

14. A method as claimed in claim 12 wherein the sub-groups are tested in parallel and at least the sub-group having the lower disease severity is tested with additional physiological outcome measures.

15. A method as claimed in claim 14 wherein the physiological outcome measures are neurophysiological outcome measures as determined using analysis of changes in functional brain scans such as to detect therapeutic efficacy even in the absence of clinical benefit as measured psychometrically.

16. A method as claimed in claim 15 wherein the functional brain scan is performed using Single Photon Emission Tomography (SPECT) or Positron Emission Tomography (PET), optionally using Region of Interest (ROI) Analysis or Statistical parametric (SPM) analysis.

17. A method as claimed in claim 15 wherein the subjects are scanned at or shortly before the time of randomisation and one or more later scans are then made after or during treatment.

18. A method as claimed in claim 1 wherein, for at least the sub-group having the higher disease severity, a linear imputation method for each individual discontinuing treatment is used to correct the analysis for the effect of non-random withdrawal of subjects randomised to the placebo or a minimal efficacy comparator treatment arm, thereby preventing or confounding the demonstration of therapeutic efficacy.

19. A method as claimed in claim 18 wherein psychometric outcome measures are made of the subjects and the linear imputation analysis is performed on the available psychometric scores of individual subjects discontinuing treatment by use of a straight line per-subject extrapolation fitted to the graph of said scores.

20. A method as claimed in claim 1 wherein the method or system constitutes a clinical trial or system for performing a clinical trial for testing the pharmaceutical.

21. A method as claimed in claim 1 wherein the method or system is to assess a treatment regime employing the pharmaceutical for its efficacy.

22. A method as claimed in claim 1 wherein the pharmaceutical is a 3,7-diaminophenothiazine (DAPTZ) compound.
